SEQUENCE LISTING (5 sequences)

SEQ ID NO: 1 - protein, 449 aa, Homo sapiens
  BINDING (1)..(119): HC Variable Region
QVQLVESGGGVVQPGRSRRLSCAASGFTFSRYTMHWVRQAPGKGLEWVAVISFDGSNKYYVDSVKGRFTISRDNSENTLYLQVNILRAEDTAVYYCAREARGSYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, Homo sapiens
  BINDING (1)..(108): LC Variable Region
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 231 aa, Homo sapiens
  BINDING (1)..(119): Heavy chain variable domain
QVQLVESGGGVVQPGRSRRLSCAASGFTFSRYTMHWVRQAPGKGLEWVAVISFDGSNKYYVDSVKGRFTISRDNSENTLYLQVNILRAEDTAVYYCAREARGSYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPGK

SEQ ID NO: 4 - protein, 275 aa, Artificial
  Construct comprising human immunogobulin regions and artificalsequences
MKYLLPTAAAGLLLLAAQPAMAQVQLVESGGGVVQPGRSLRLSCAASGFTFSRYTMHWVRQAPGKGLEWVAVISFDGSNKYYVDSVKGRFTISRDNSENTLYLQVNILRAEDTAVYYCAREARGSYAFDIWGQGTMVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIKHHHHHHGGGGC

SEQ ID NO: 5 - DNA, 893 bp, Artificial
  human codons replaced with E. coli preferred codons
gaattctatggaacgataaatgcccatgaaaattctatttcaaggagacagtcataatgaaatacctgctgccgaccgctgctgctggtctgctgctgctggcggcccagccggccatggctcaggtgcagctggtggagtctggtggtggcgtggtccagcctggtcgttcccgtcgcctctcctgtgcagcctctggtttcaccttcagtcgttatactatgcactgggtccgccaggctccaggcaagggtctggagtgggtggcagttatctcatttgatggtagcaacaaatactacgtagactccgtgaagggccgtttcaccatctcccgtgacaactccgagaacacgctgtatctgcaagtgaacatcctgcgtgctgaggacacggctgtgtattactgtgcgcgtgaggcccgtggttcctatgcttttgatatctggggccaaggtaccatggtcaccgtctcttccggtggcggtggctccggtggcggtggctccggtggcggtggatccgaaattgtgctgacccagtctccagccaccctgtctctgtctccaggtgaacgtgccaccctctcctgccgtgccagtcagagtgttagcagctacctggcctggtaccaacagaaacctggccaggctccgcgtctcctcatctatgatgcatccaaccgtgccactggcatcccagcccgtttcagtggcagtggttctggtaccgacttcactctcaccatcagcagcctggagcctgaagattttgcagtttattactgtcagcagcgtagcaactggcctccattcactttcggccctggtaccaaagtggatatcaaacaccaccatcaccaccatggtggtggtggttgctaataactcgag

(19) United States
(12) Patent Application Publication
(10) Pub. No.: US 2009/0163698 A1
(43) Pub. Date: Jun. 25, 2009

(54) Method for Preparing Antibody Conjugates

(76) Inventor: John Joseph Grigsby, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/119,025

(22) Filed: May 12, 2008

Related U.S. Application Data
(60) Provisional application No. 60/917,614, filed on May 11, 2007.

(51) Int. Cl. C07K 16/00

(52) U.S. Cl. 530/387.1

(57) ABSTRACT

The subject matter described herein relates to a process for preparing an antibody, in one embodiment a Fab′, in very good yields and purity, under conditions where the presence of heavy and light chain antibody fragments is minimized. More particularly, the subject matter described herein relates to a process for reducing F(ab)² to primarily heavy and light chains, followed by a reoxidation step that is selective for making Fab′ in very good yields and purity by reforming the disulfide bridge between the heavy and light chains. The reoxidation step is carried out to minimize the presence of heavy and light chain, minimize the generation of F(ab′)2 and maximize Fab′. In one embodiment, the subject matter described herein relates to a process for preparing an antibody composition, in one embodiment a Fab′ liposome composition, having specific binding activity for alpha-V-integrin receptors. The composition is intended for use in treating conditions characterized by cells that express any alpha-V-comprising integrin, such as αvβ3, αvβ5, and αvβ6 receptors.

### CROSS-REFERENCE TO RELATED APPLICATION

This application claims priority to U.S. Provisional application No. 60/917,614, filed 11 May 2007, the entire contents of which is incorporated herein by reference.

### FIELD

The subject matter described herein relates to a process for preparing an antibody, in one embodiment a Fab′, in very good yields and purity, under conditions where the presence of heavy and light chain antibody fragments is minimized. More particularly, the subject matter described herein relates to a process for reducing F(ab)² to primarily heavy and light chains, followed by a reoxidation step that is selective for making Fab′ in very good yields and purity by reforming the disulfide bridge between the heavy and light chains. The reoxidation step is carried out to minimize the presence of heavy and light chain, minimize the generation of F(ab′)2 and maximize Fab′. In one embodiment, the subject matter described herein relates to a process for preparing an antibody composition, in one embodiment a Fab′ liposome composition, having specific binding activity for alpha-V-integrin receptors. The composition is intended for use in treating conditions characterized by cells that express any alpha-V-comprising integrin, such as αvβ3, αvβ5, and αvβ6 receptors.

### BACKGROUND

Integrins are a superfamily of cell adhesion receptors, which exist as heterodimeric transmembrane glycoproteins. They are part of a large family of cell adhesion receptors which are involved in cell-extracellular matrix and cell-cell interactions. Integrins play critical roles in cell adhesion to the extracellular matrix (ECM) which, in turn, mediates cell survival, proliferation and migration through intracellular signaling. The receptors consist of two subunits that are non-covalently bound. Those subunits are called alpha (α) and beta (β). The alpha subunits all have some homology to each other, as do the beta subunits. The receptors always contain one alpha chain and one beta chain and are thus called heterodimeric. Both of the subunits contribute to the binding of the ligand. Eighteen alpha subunits and eight beta subunits have been identified, which heterodimerize to form at least 24 distinct integrin receptors.

Among the variety of alpha chain subunits is a protein chain referred to as alpha-V. The ITGAV gene encodes integrin alpha chain V (vitronectin receptor, alpha-v; αv, antigen CD51). The I-domain containing integrin alpha-V undergoes post-translational cleavage to yield disulfide-linked heavy and light chains, that combine with multiple integrin beta chains to form different integrins. Alternative splicing of the gene yields seven different transcripts; a, b, c, e, f, h, j altogether encoding six different protein isoforms of alpha-V. Among the known associating beta chains (beta chains 1, 3, 5, 6, and 8; ‘ITGB1’, ‘ITGB3’, ‘ITGB5’, ‘ITGB6’, and ‘ITGB8’), each can interact with extracellular matrix ligands. The alpha V beta 3 integrin, perhaps the most studied of these, is referred to as the vitronectin receptor (VNR). In addition to providing for cell attachment to other cells or to extracellular proteins such as vitronectin (αvβ3) and fibronectin (αvβ6), the integrins are capable of intracellular signaling which provides clues for cell migration and secretion of or elaboration of other proteins involved in cell motility and invasion and angiogenesis. The alpha-V integrin subfamily of integrins recognize the ligand motif arg-gly-asp (RGD) present in fibronection, vitronection, VonWillebrand factor, and fibrinogen. The alpha-V integrins are receptors for vitronectin, cytotactin, fibronectin, fibrinogen, laminin, matrix metalloproteinase-2, osteopontin, osteomodulin, prothrombin, thrombospondin and von Willebrand factor. In case of HIV-1 infection, the interaction with extracellular viral Tat protein seems to enhance angiogenesis in Kaposi's sarcoma lesions.

It has been established that integrins which are alpha-V containing heterodimers, particularly alpha-V-/beta-6, the receptor for fibronectin, are involved in adhesion of carcinoma cells to fibronectin and vitronectin. This is especially true for carcinoma cells arising from the malignant progression of colon cancer (Lehmann, M. et al., Cancer Res., 54(8):2102-7 (1994)). Furthermore, integrin expression in colon cancer cells is regulated by the cytoplasmic domain of the beta-6 integrin subunit which signals through the ERK2 pathway (Niu, J. et al., Int. J. Cancer, 99(4):529-537 (2002)) and beta6 expression is associated with secretion of gelatinase B. an enzyme involved in tumor cell invasion and metastatic mechanisms (Agrez et al., Int. J. Cancer, 81(1):90-97 (1999)).

There is now considerable evidence that progressive tumor growth is dependent upon angiogenesis, the formation of new blood vessels, to provide tumors with nutrients and oxygen, to carry away waste products and to act as conduits for the metastasis of tumor cells to distant sites (Gastl, G. et al., Oncol., 54(3):177-184 (1997)). Recent studies have further defined the roles of integrins in the angiogenic process. During angiogenesis, a number of integrins that are expressed on the surface of activated endothelial cells regulate critical adhesive interactions with a variety of ECM proteins to regulate distinct biological events such as cell migration, proliferation and differentiation. Specifically, the closely related but distinct integrins αvβ3 and αvβ5 have been shown to mediate independent pathways in the angiogenic process. An antibody generated against αvβ3 blocked basic fibroblast growth factor (bFGF) induced angiogenesis, whereas an antibody specific to αvβ5 inhibited vascular endothelial growth factor (VEGF) induced angiogenesis (Eliceiri et al., J. Clin. Invest., 103:1227-1230 (1999); Friedlander et al., Science, 270:1500-1502 (1995)). Therefore, integrins, and especially the alpha-V subunit containing integrins, are a therapeutic targets for diseases that involve angiogenesis, such as diseases of the eye and neoplastic diseases, tissue remodeling such as restenosis, and proliferation of certain cells types, particularly epithelial and squamous cell carcinomas.

The use of antibodies for treating human diseases, such as the diseases that involve angiogenesis and mediated by integrins, is well established and has become more sophisticated with the introduction of genetic engineering. Several techniques have been developed to improve the utility of antibodies. These include: (1) the generation of monoclonal antibodies by cell fusion to create “hybridomas”, or by molecular cloning of antibody heavy and light chains from antibody-producing cells; (2) the conjugation of other molecules to antibodies to deliver them to preferred sites in vivo, e.g., radioisotopes, toxic drugs, protein toxins, and cytokines; (3) the manipulation of antibody effector functions to enhance or diminish biological activity; (4) the joining of other proteins such as toxins and cytokines with antibodies at the genetic level to produce antibody-based fusion proteins; and (5) the joining of one or more sets of antibody combining regions to lipids or lipopolymers.

Antibodies, such as those that target integrin receptors, can be administered to a subject in need of treatment, or alternatively the antibodies can be combined with a therapeutic agent to form a therapeutic antibody composition. The antibody can be combined or conjugated directly to the therapeutic agent or to a delivery vehicle that contains a therapeutic agent, and then administered to a subject in need of treatment. One such delivery vehicle is a lipitic microparticle like a liposome or a lipid-based component of a liposome (see, for example, U.S. Pat. No. 6,210,707 which is incorporated herein by reference, for discussion of lipidic microparticles).

Liposomes are spherical vesicles comprised of concentrically ordered lipid bilayers that encapsulate an aqueous phase. Liposomes serve as a delivery vehicle for therapeutic agents contained in the aqueous phase or in the lipid bilayers. Delivery of drugs in liposome-entrapped form can provide a variety of advantages, depending on the drug, including, for example, a decreased drug toxicity, altered pharmacokinetics, or improved drug solubility. Liposomes when formulated to include a surface coating of hydrophilic polymer chains, so-called Stealth® liposomes, offer the further advantage of a long blood circulation lifetime, due in part to reduced removal of the liposomes by the mononuclear phagocyte system. Often an extended lifetime is necessary in order for the liposomes to reach their desired target region or cell from the site of injection.

Targeted liposomes have targeting ligands or affinity moieties attached to the surface of the liposomes. The targeting ligands may be antibodies or fragments thereof, in which case the liposomes are referred to as immunoliposomes. When administered systemically targeted liposomes deliver the entrapped therapeutic agent to a target tissue, region or, cell. Because targeted liposomes are directed to a specific region or cell, healthy tissue is not exposed to the therapeutic agent. Such targeting ligands can be attached directly to the liposomes' surfaces by covalent coupling of the targeting ligand to the polar head group residues of liposomal lipid components (see, for example, U.S. Pat. No. 5,013,556 which is incorporated herein by reference). This approach, however, is suitable primarily for liposomes that lack surface-bound polymer chains, as the polymer chains interfere with interaction between the targeting ligand and its intended target (Klibanov, A. L., et al., Biochim. Biophys. Acta., 1062:142-148 (1991); Hansen, C. B., et al., Biochim. Biophys. Acta, 1239:133-144 (1995)).

Alternatively, the targeting ligands can be attached to the free ends of the polymer chains forming the surface coat on the liposomes (Allen. T. M., et al., Biochim. Biophys. Acta, 1237:99-108 (1995); Blume, G. et al., Biochim. Biophys. Acta, 1149:180-184 (1993)). In this approach, the targeting ligand is exposed and readily available for interaction with the intended target.

Antibody immunoliposomes, such as Fab′ immunoliposomes, have been prepared by conjugating Fab′ to a liposome, or liposomal component that is used in the preparation of the liposome. Reducing agents are known to reduce F(ab)² to Fab′, such as cysteine and mercaptoethylamine, or dithiothreitol. Shahinian et al., Biochim Biophys Acta, 1239(2):157-67 (1995 Nov. 1).

Unfortunately, the process of obtaining Fab′ from F(ab)² is complex and tends to be very specific. In certain cases, reduction of the F(ab)² to Fab′ is not very selective and the reduction process breaks the disulfide bridges between heavy and light chains. If the goal of immunoliposome production is the preparation of Fab′ immunoliposomes, it can be understood that the presence of heavy and light chains in the conjugation process is unwelcome since the broken disulfide bridges allow the heavy and light chains to undergo the conjugation process and form impurities. Purification of the heavy and light chains from Fab′ prior to conjugation is not feasible because of the noncovalent interactions that continue to hold the heavy and light chains together, thus encumbering the separation of the heavy and light chains from Fab′. Therefore, unless heavy and light chains are removed from the Fab′ preparation, the heavy and light chains will be taken through the conjugation process along with the Fab′. Post conjugation separation of heavy and light chain conjugates from Fab conjugates is difficult because they form micelles, and the purification of the Fab conjugates would likely require the use of denaturants to break the interactions between the heavy and light chains which raises additional concerns such as protein refolding.

Therefore, in light of the foregoing, it can be understood that what is needed is a process for preparing an antibody, in one embodiment a Fab′, in very good yields and purity, under conditions where the presence of heavy and light chain antibody fragments is minimized. More particularly, what is needed is a process for reducing F(ab)² to heavy and light chain followed by a reoxidation step that is selective for making Fab′, in very good yields and purity, by reforming the disulfide bridge between the heavy and light chains. The reoxidation step should be carried out to minimize the presence of heavy and light chain, minimize the generation of F(ab′)2 and maximize Fab′.

The foregoing examples of the related art and limitations related therewith are intended to be illustrative and not exclusive. Other limitations of the related art will become apparent to those of skill in the art upon a reading of the specification and a study of the drawings.

### SUMMARY

The following aspects and embodiments thereof described and illustrated below are meant to be exemplary and illustrative, not limiting in scope.

The subject matter described herein relates to a process for preparing an antibody with very good purity and yield while minimizing the amount of heavy and light chains present.

In one embodiment, a process is disclosed for reducing an antibody, such as F(ab)₂, to heavy and light chains followed by a reoxidation step that is selective for making Fab′ in very good yields and purity by reforming the disulfide bridge between the heavy and light chains. The reoxidation step is carried out to minimize the presence of heavy and light chains, minimize the generation of F(ab′)2 and maximize Fab′. The oxidation step to form Fab′ from heavy and light chains is performed prior to conjugation to a therapeutic agent or a delivery vehicle such as a liposome or a liposomal component. This minimizes the amount of heavy and light chain conjugates in the final product and eliminates the need to separate out the heavy and light chain conjugated species.

In another aspect, a process is disclosed for preparing immuno drug conjugates, comprising a Fab′, wherein the Fab′ is prepared in high yield and purity through a process that minimizes the amount of heavy and light chains present. The Fab′ is conjugated to a therapeutic agent, generally any therapeutic agent that is capable of attaching by itself or through a linker to at least one sulfhydryl end of the Fab′.

In another aspect, a process is disclosed for preparing immuno lipidic microparticle conjugates, comprising a Fab′, wherein the Fab′ is prepared in high yield and purity through a process that minimizes the amount of heavy and light chains present. The Fab′ is conjugated to a lipid, or a lipidic microparticle such as a lipopolymer.

In another aspect, a process is disclosed for preparing immunoliposomes conjugates, comprising a Fab′ and a lipid or lipopolymer, wherein the Fab′ is prepared in high yield and purity through a process that minimizes the amount of heavy and light chains present. The Fab′ is conjugated to a lipid or lipopolymer that comprises a liposomal composition.

In one embodiment, a lipopolymer with a reactive end is inserted into drug loaded liposomes, for subsequent conjugation between the reactive end of the lipopolymer and a Fab′. The Fab′ is prepared by first reducing an antibody, such as F(ab)², to cleave solvent accessible disulfide bonds and then reoxidizing the protein in a controlled manner to selectively reform the disulfide bonds between the heavy and light chains, thus forming Fab′ at a high purity. The reoxidized Fab′ is then added to the pre-formed liposomes bearing reactive groups to conjugate the Fab′ ligand to the external surface of the liposomes. The conjugation is performed at room temperature conditions (or in general at any temperature where the Fab′ is stable).

In another embodiment, a Fab′ is prepared by first reducing an antibody, such as F(ab)², to cleave solvent accessible disulfide bonds and then reoxidizing the protein in a controlled manner to selectively reform the disulfide bonds between the heavy and light chains, thus forming Fab′ at a high purity. The reoxidized Fab′ is then conjugated to a lipopolymer with a reactive end and the conjugated Fab′ lipopolymer is inserted into the bilayer of a pre-formed liposome by incubating the Fab′ lipopolymer conjugate with the pre-formed liposome. The Fab′ is preferably one that resists denaturation and retains its biological activity during insertion conditions (conditions generally requiring higher temperatures above the phase transition temperature of the lipids).

In another aspect, a process is provided for preparing an immunoliposome composition as described above for targeting to a human alpha-V integrin subunit. In another aspect, a process for preparing an immunoliposome composition as described above capable of specific binding to a cell expressing alpha-V integrin is provided.

In another aspect, a process is provided for preparing an alpha-V-targeting immunoliposome composition as described above comprised of liposomes bearing a targeting ligand which is an antibody-derived construct, such as an antibody fragment or derivative, for targeting to a human alpha-V integrin subunit.

In another embodiment, a process is provided for preparing the aforementioned alpha-V-targeting immunoliposome composition, the targeting ligand comprised of a heavy chain variable region derived from a parent antibody capable of specific binding to at least one of alpha-V-beta1, alpha-V-beta3, alpha-V-beta5, alpha-V-beta6, alpha-V-beta8. In a specific embodiment the targeting ligand comprises the antibody heavy chain variable region residues 1-119 of SEQ ID NO: 1 comprising a framework-1 (FR1), complementarity determining region 1 (CDR1), FR2, CDR2, FR3, CDR3 and FR4 sequences. In one embodiment, the targeting ligand is comprised of a light chain variable region residues 1-108 of SEQ ID NO: 2 comprising FR1, CDR1, FR2, CDR2, FR3, CDR3 and FR4 sequences.

In still another aspect, a process is provided for preparing the aforementioned alpha-V-targeting immunoliposome composition, the targeting ligand comprised of antibody heavy and light chain variable region having a sequence identified as SEQ ID NO: 1 residues 1-119 and SEQ ID NO: 2, residues 1-108.

In these various embodiments, the alpha-V-targeting immunoliposome includes an active entrapped in the liposomes, where ‘entrapped’ intends associated with the liposome lipid bilayer or with the internal aqueous compartments. The agent, in one embodiment, is a therapeutic agent, such as an antineoplastic agent. In a specific embodiment, the antineoplastic is a cytotoxic or cytostatic agent, such as doxorubicin.

In another embodiment, products prepared by the foregoing processes are disclosed. The products prepared include, but are not limited to a Fab′ preparation where heavy and light chains are minimized, a Fab′ immuno drug conjugate, a Fab′ immuno lipopolymer conjugate, and a Fab′ targeted liposome.

In addition to the exemplary aspects and embodiments described above, further aspects and embodiments will become apparent by reference to the drawings and by study of the following descriptions.

### BRIEF DESCRIPTION OF THE DRAWINGS

FIGS. 1A-1D are images, obtained using a confocal microscope, of A375S.2 cells incubated at 4° C. for 30 minutes with liposomes containing a fluorescent marker, where FIG. 1A-1B correspond to cells incubated with liposomes lacking a targeting ligand, and FIGS. 1C-1D are images of cells incubated with liposomes bearing alpha-integrin Fab targeting ligands (90:1 Fab:liposome); FIGS. 2A-2H are images, obtained using a confocal microscope, of A375.S2 cells incubated at 37° C. for 10 minutes with liposomes containing a fluorescent marker, washed and incubated for 1 hours at 37° C., where the images correspond to untreated cells (FIGS. 2A-2B), cells treated with free doxorubicin (FIGS. 2C-2D), cells treated with liposomes lacking a targeting ligand (FIGS. 2E-2F), and cells incubated with liposomes bearing alpha-integrin Fab targeting ligands (90:1 Fab:liposome, FIGS. 2G-2H);

FIGS. 3A-3J are images, obtained using a confocal microscope, of A375.S2 cells incubated at 37° C. for 10 minutes with liposomes containing a fluorescent marker, washed and incubated for 0, 6, or 24 hours at 37° C., where the images correspond to untreated cells (FIGS. 3A-3B), cells treated with liposomes bearing alpha-integrin Fab targeting:ligands (90:1 Fab:liposome) and incubated for 0 hours (FIGS. 3C-3D), 6 hours (FIGS. 3E-3F), 24 hours (FIGS. 3G-3H), or with liposomes lacking a targeting ligand (FIGS. 31-3J; 24 hour post-wash incubation);

FIGS. 4A-4H are images, obtained using a confocal microscope, of B16-F10 cells incubated at 37° C. for 10 minutes with liposomes containing doxorubicin, washed and incubated for 1 hours at 37° C., where the images correspond to untreated cells (FIGS. 4A-5B), cells treated with free doxorubicin (FIGS. 4C-4D), cells treated with liposomes lacking a targeting ligand (FIGS. 4E-4F), and cells incubated with liposomes bearing alpha-integrin Fab targeting ligands (90:1 Fab:liposome, FIGS. 4G-4H);

FIGS. 5A-5C are graphs showing the percent of viable A375.S2 cells, expressed as a percent of untreated control cells, as a function of doxorubicin concentration, in μg/mL, the doxorubicin in free form (squares), entrapped in liposomes lacking a targeting ligand (triangles), entrapped in liposomes bearing alpha-integrin Fab targeting ligands at Fab:liposome ratios of 15:1 (x symbols), 40:1 (FIG. 5A, diamonds), 90:1 (FIG. 5B, diamonds; FIGS. 5A-5C, * symbols), 180:1 (FIG. 5C, diamonds);

FIGS. 6A-6B are graphs showing the percent of viable MDA-MB-231 cells, expressed as a percent of untreated control cells, as a function of doxorubicin concentration, in μg/mL, the doxorubicin in free form (squares), entrapped in liposomes lacking a targeting ligand (triangles), entrapped in liposomes bearing alpha-integrin Fab targeting ligands at Fab:liposome ratios of 15:1 (x symbols), 40:1 (FIG. 7A, diamonds; FIG. 6B, circles), and 90:1 (* symbols);

FIG. 7 is a graph showing the percent of viable A2780 cells, expressed as a percent of untreated control cells, as a function of doxorubicin concentration, in μg/mL, the doxorubicin in free form (squares), entrapped in liposomes lacking a targeting ligand (triangles), entrapped in liposomes bearing alpha-integrin Fab targeting ligands at Fab:liposome ratios of 15:1 (diamonds), 40:1 (* symbols), and 90:1 (circles);

FIG. 8 is a graph showing the percent of viable B16-F10 cells, expressed as a percent of untreated control cells, as a function of doxorubicin concentration, in μg/mL, the doxorubicin in free form (squares), entrapped in liposomes lacking a targeting ligand (triangles), or entrapped in liposomes bearing alpha-integrin Fab targeting ligands at Fab:liposome ratios of 90:1 (diamonds);

FIG. 9 is a graph showing reoxidation reproducibility based on gel quantitation.

FIG. 10 is a graph showing Fab conjugation efficiency reproducibility based on gel quantitation.

FIG. 11 is a graph showing Fab conjugation purity reproducibility based on gel quantitation.

FIG. 12 is a graph showing Fab conjugation yield reproducibility based on gel quantitation.

### BRIEF DESCRIPTION OF THE SEQUENCES

| SEQ ID NO: | Description | Features |
|---|---|---|
| 1 | Parent antibody: heavy chain | |
| 2 | Parent antibody: light chain | |
| 3 | Secreted Fab heavy chain | |
| 4 | Single Chain antibody derived from parent | |
| | antibody | |
| 5 | Nucleic acid construct for expression of scFv | |
| | in E. coli | |

### DETAILED DESCRIPTION

### I. Definitions & Abbreviations

Abbreviations CV column volume; Fv, antibody variable fragment consisting of VH and VL; scFv, single chain variable fragment; VH, variable heavy; VL, Variable light; PEG, Polyethylene Glycol; Gly4Cys, four glycine residues followed by a cysteine residue; H is Tag, six histidine amino acid residues at the C-terminus of the protein; Fc, Fragment crystallizable

### DEFINITIONS

The term “alpha-V (αv) integrin”, “alpha-V subunit integrin”, and “alpha-V subunit containing integrin” are used interchangeably herein to mean alpha-V transmembrane glycoprotein subunits of a functional integrin heterodimer and include all of the variants, isoforms and species homologs of alpha-V. Alpha-V polypeptides include one or more isoforms of proteins encoded by the ITGAV gene having names integrin, alpha-V (vitronectin receptor, alpha polypeptide, antigen CD51); other aliases include, CD51, MSK8, VNRA; and other designations are integrin, alpha-V (vitronectin receptor, alpha polypeptide); antigen identified by monoclonal antibody L230; integrin alpha-V. The gene is located on human chromosome 2; location: 2q31-q32 (MIM: 193210; GeneID: 3685) The alpha-V-comprising integrins bind a wide variety of ligands. Human antibodies may, in certain cases, cross-react with alpha-V from species other than human, or other proteins that are structurally related to human alpha-V (e.g., human alpha v homologs). In other cases, the antibodies may be completely specific for human alpha-V and not exhibit species or other types of cross-reactivity.

As used herein, an “antibody” includes whole antibodies and any antigen binding fragment or single chain fragment thereof. Thus the antibody includes any protein or peptide containing molecule that comprises at least a portion of an immunoglobulin molecule, such as but not limited to at least one complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, which can be incorporated into an antibody that is prepared in accordance with the processes disclosed herein. An “alpha-V antibody”, “alpha-V subunit antibody” or “alpha-V integrin antibody” is an antibody that specifically binds the alpha-V subunit of an integrin. The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian alpha-V subunit. Examples of binding fragments encompassed within the term “antigen binding portion” of an antibody include (i) a Fab fragment, a monovalent fragment consisting of the VL, VH, CL and CH, domains; (ii) a F(ab′)₂ fragment, a bivalent fragment comprising two Fab fragments linked by a disulfide bridge at the hinge region; (iii) a Fd fragment consisting of the VH and CH, domains; (iv) a Fv fragment consisting of the VL and VH domains of a single arm of an antibody, (v) a dAb fragment (Ward et al., Nature, 341:544-546 (1989)), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. Science, 242:423-426 (1988), Huston et al., Proc. Natl. Acad. Sci. USA, 85:5879-5883 (1988)). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the CH₁ domain and/or hinge region of the heavy chain. The various portions of antibodies can be joined together chemically by conventional techniques, or can be prepared as a contiguous protein using genetic engineering techniques.

A “complementarity determining region” or “CDR” refers to regions of somatic hypermutation of the immunoglobulin variable genes which occur after antigen stimulation during the differentiation of the B lymphocyte in the lymph glands leading to an amino acid sequence in the variable region of an antibody which impart the affinity and specificity of binding to the antibody; positioned at the end of several looped structures within the variable domain, CDRs form a surface that is “complementary to” the surface of an antigen or an epitope of that antigen.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Epitopes resulting from conformational folding of the integrin molecule which arise when amino acids from differing portions of the linear sequence of the integrin molecule come together in close proximity in three-dimensional space. Such conformational epitopes are distributed on the extracellular side of the plasma membrane. Conformational and nonconformational epitopes are distinguished in that the binding to the former but not the latter is lost in the presence of denaturing solvents.

A “framework region” or FR” refers to amino acid sequences which are found between complementarity determining regions (CDRs) in an antibody variable domain and are derived from the germline Heavy chain Variable (IGHV) genes (V, D, J genes) sequences of the human antibody genes.

Unless otherwise noted, the term “incubating” refers to conditions of time, temperature and liposome lipid composition which allow for penetration and entry of a selected component, such as a lipid or lipid conjugate, into the lipid bilayer of a liposome.

Unless otherwise noted, the term “pre-formed liposomes” refers to intact, previously formed unilamellar or multilamellar lipid vesicles.

Unless otherwise noted, the term “sensitized to a cell” or “target-cell sensitized” refers to a liposome that includes a ligand or affinity moiety covalently bound to the liposome and having binding affinity for alpha-V-beta3 (αvβ3) and alpha-V-beta5 (αvβ5) receptor expressed or other alpha-V subunit-containing integrins on a cell.

Unless otherwise noted, the term “therapeutic liposome composition” refers to liposomes that include a therapeutic agent entrapped in the aqueous spaces of the liposomes or in the lipid bilayers of the liposomes.

Unless otherwise noted, the term “vesicle-forming lipid” refers to any lipid capable of forming part of a stable micelle or liposome composition and typically including one or two hydrophobic, hydrocarbon chains or a steroid group and may contain a chemically reactive group, such as an amine, acid, ester, aldehyde or alcohol, at its polar head group.

The term “human antibody”, as used herein, is intended to include antibodies having variable and constant regions derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germine of another mammalian species, such as a mouse, have been grafted onto human framework sequences. Thus as used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

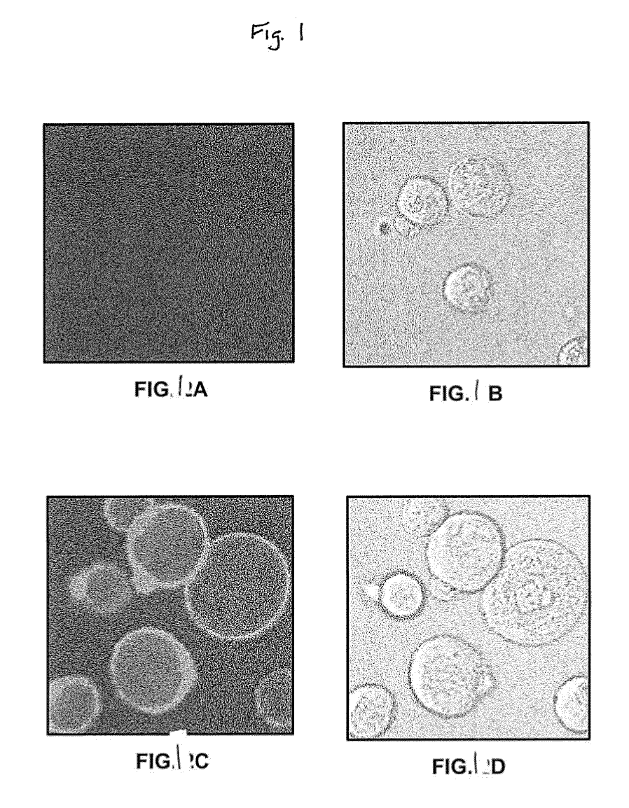

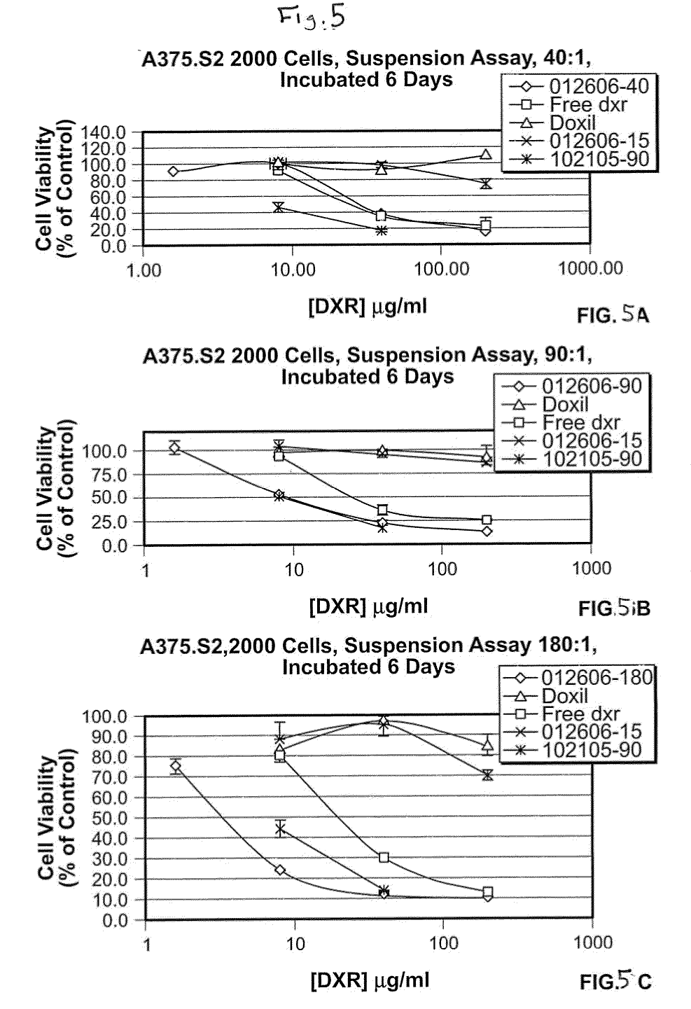

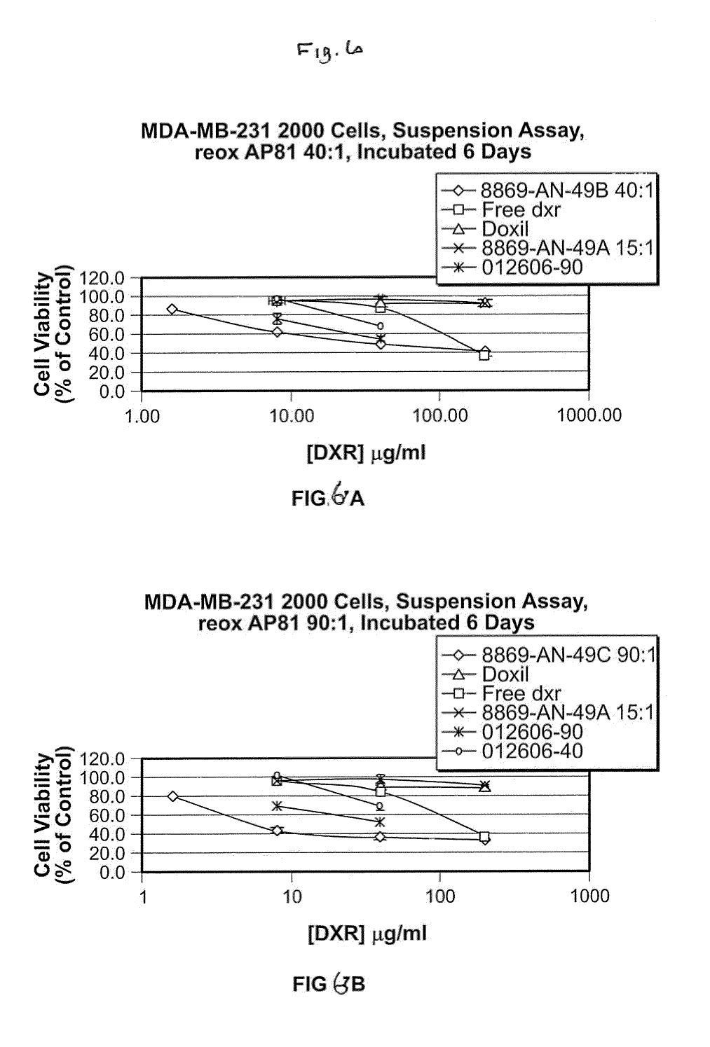

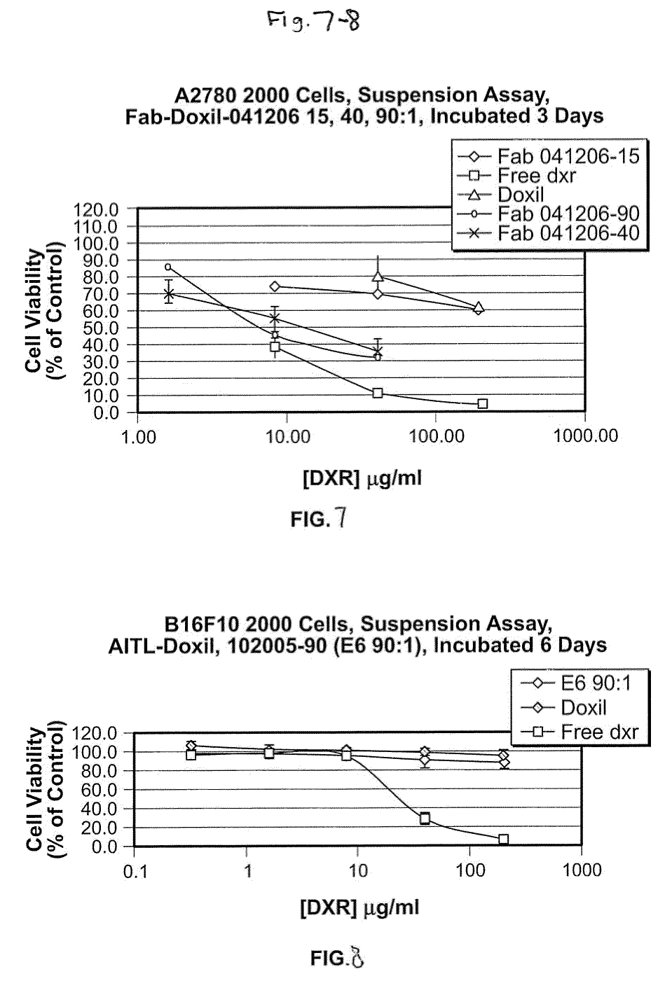

As used herein, a human antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. Human germline antibody consensus sequences for various regions and domains of human antibodies; FR1, FR2, FR3, FR4, CH1, hinge1, hinge2, hinge 3, hinge4, CH2, CH3 or fragment thereof are described in, and optionally with at least one substitution, insertion or deletion as provided in FIGS. 1-42 of, PCT WO05/005604 and U.S. Ser. No. 10/872,932 each entirely incorporated herein by reference. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germine sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than ten amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

A monoclonal antibody from a non-human animal, such as a mouse, rat, baboon, or rabbit, may also be used as a parent antibody providing a source of the alpha-V binding regions of the antibody-derived targeting-ligand.

The terms “monoclonal antibody” or “parental antibody” as used herein refer to a preparation of antibody molecules of single molecular composition. A monoclonal antibody displays a single binding specificity and affinity for a particular epitope. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

An “isolated antibody,” as used herein, is intended to refer to an antibody which is substantially free of other antibodies having different antigenic specificities (e.g., an isolated antibody that specifically binds to alpha-V is substantially free of antibodies that specifically bind antigens other than alpha-V). An isolated antibody that specifically binds to an epitope, isoform or variant of human alpha-V may, however, have cross-reactivity to other related antigens, e.g., from other species (e.g., alpha-V species homologs). Moreover, an isolated antibody may be substantially free of other cellular material and/or chemicals.

As used herein, “specific binding” refers to antibody binding to a predetermined antigen. Typically, the parental antibody binds with a dissociation constant (K_{D}) of 10⁻⁷ M or less, and binds to the predetermined antigen with a K_{D} that is at least twofold less than its K_{D} for binding to a non-specific antigen (e.g., BSA, casein) other than the predetermined antigen or a closely-related antigen.

The phrases “an antibody recognizing an antigen” and “an antibody specific for an antigen” are used interchangeably herein with the term “an antibody which binds specifically to an antigen”.

As used herein, K_{D} refers to the dissociation constant, specifically, the antibody K_{D} for a predetermined antigen, and is a measure of affinity of the antibody for a specific target. High affinity antibodies have a K_{D} of 10⁻⁸ M or less, more preferably 10⁻⁹ M or less and even more preferably 10⁻¹¹ M or less, for a predetermined antigen. The reciprocal of K_{D} is K_{A}, the association constant. The term “k_{dis}” or “k₂”, or “k_{d}”, is intended to refer to the dissociation rate of a particular antibody-antigen interaction. The “K_{D}”, is the ratio of the rate of dissociation (k₂), also called the “off-rate (k_{off})”, to the rate of association rate (k₁) or “on-rate (kₒₙ)”. Thus, K_{D} equals k₂/k₁ or k_{off}/kₒₙ and is expressed as a molar concentration (M). It follows that the smaller the K_{D}, the stronger the binding. So a K_{D} of 10⁻⁶ M (or 1 microM) indicates weak binding compared to 10⁻⁹ M (or 1 nM).

### II. Antibodies and Immunolipidic Microparticles

The subject matter described herein relates to a process for preparing an antibody with very good purity and yield while minimizing the amount of heavy and light chains present.

In one embodiment, a process is disclosed for reducing an antibody, such as F(ab)₂, to heavy and light chains followed by a reoxidation step that is selective for making Fab′ in very good yields and purity by reforming the disulfide bridge between the heavy and light chains. The reoxidation step is carried out to minimize the presence of heavy and light chains, minimize the generation of F(ab′)² and maximize Fab′. The oxidation step to form Fab′ from heavy and light chains is performed prior to conjugation to a therapeutic agent or a delivery vehicle such as a liposome or a liposomal component. This minimizes the amount of heavy and light chain conjugates in the final product and eliminates the need to separate out the heavy and light chain conjugated species.

In another aspect, a process is disclosed for preparing immuno drug conjugates, comprising a Fab′, wherein the Fab′ is prepared in high yield and purity through a process that minimizes the amount of heavy and light chains present. The Fab′ is conjugated to a therapeutic agent, generally any therapeutic agent that is capable of attaching by itself or through a linker to at least one sulfhydryl end of the Fab′.

In another aspect, a process is disclosed for preparing immuno lipidic microparticle conjugates, comprising a Fab′, wherein the Fab′ is prepared in high yield and purity through a process that minimizes the amount of heavy and light chains present. The Fab′ is conjugated to a lipid, or a lipidic microparticle such as a lipopolymer.

In another aspect, a process is disclosed for preparing immunoliposomes conjugates, comprising a Fab′ and a lipid or lipopolymer, wherein the Fab′ is prepared in high yield and purity through a process that minimizes the amount of heavy and light chains present. The Fab′ is conjugated to a lipid or lipopolymer that comprises a liposomal composition.

In one embodiment, a lipopolymer with a reactive end is inserted into drug loaded liposomes, for subsequent conjugation between the reactive end of the lipopolymer and a Fab′. The Fab′ is prepared by first reducing an antibody, such as F(ab)², to cleave solvent accessible disulfide bonds and then reoxidizing the protein in a controlled manner to selectively reform the disulfide bonds between the heavy and light chains, thus forming Fab′ at a high purity. The reoxidized Fab′ is then added to the pre-formed liposomes bearing reactive groups to conjugate the Fab′ ligand to the external surface of the liposomes. The conjugation is performed at room temperature conditions (or in general at any temperature where the Fab′ is stable).

In another embodiment, a Fab′ is prepared by first reducing an antibody, such as F(ab)², to cleave solvent accessible disulfide bonds and then reoxidizing the protein in a controlled manner to selectively reform the disulfide bonds between the heavy and light chains, thus forming Fab′ at a high purity. The reoxidized Fab′ is then conjugated to a lipopolymer with a reactive end and the conjugated Fab′ lipopolymer is inserted into the bilayer of a pre-formed liposome by incubating the Fab′ lipopolymer conjugate with the pre-formed liposome (see, for example, U.S. Pat. No. 6,210,707 which is incorporated herein by reference). In one embodiment, the Fab′ is one that resists denaturation and retains its biological activity during insertion conditions (conditions generally requiring higher temperatures to allow the phase transition of the lipids).

In another aspect, a process is provided for preparing an immunoliposome composition as described above for targeting to a human alpha-V integrin subunit. In another aspect, a process for preparing an immunoliposome composition as described above capable of specific binding to a cell expressing alpha-V integrin is provided.

In another aspect, a process is provided for preparing an alpha-V-targeting immunoliposome composition as described above comprised of liposomes bearing a targeting ligand which is an antibody-derived construct, such as an antibody fragment or derivative, for targeting to a human alpha v integrin subunit.

In another embodiment, a process is provided for preparing the aforementioned alpha-V-targeting immunoliposome composition, the targeting ligand comprised of a heavy chain variable region derived from a parent antibody capable of specific binding to at least one of alpha-V-beta1, alpha-V-beta3, alpha-V-beta5, alpha-V-beta6, alpha-V-beta8. In a specific embodiment the targeting ligand comprises the antibody heavy chain variable region residues 1-119 of SEQ ID NO: 1 comprising a framework-1 (FR1), complementarity determining region 1 (CDR1), FR2, CDR2, FR3, CDR3 and FR4 sequences. In one embodiment, the targeting ligand is comprised of a light chain variable region residues 1-108 of SEQ ID NO: 2 comprising FR1, CDR1, FR2, CDR2, FR3, CDR3 and FR4 sequences.

In still another aspect, a process is provided for preparing the aforementioned alphaV-targeting immunoliposome composition, the targeting ligand comprised of antibody heavy and light chain variable region having a sequence identified as SEQ ID NO: 1 residues 1-119 and SEQ ID NO: 2, residues 1-108.

In another aspect, a process is provided for preparing an immunoliposome composition, the composition comprised of liposomes that include as a targeting ligand an antibody-derived protein which is a monomeric, dimeric or multimeric construct, having binding specificity for an αv-comprising integrin on the surface of a cell. The alpha-V targeting-ligand is incorporated into the liposomes in the form of a lipid-polymer-protein conjugate, also referred to herein as a lipid-polymer-ligand conjugate. As will be described below, the antibody-derived construct has specific affinity for αv-integrin receptors, and targets the liposomes to cells that express any of the alpha-V-comprising intergrin heterodimers including but not limited to αvβ3, αvβ5 and αvβ6 receptors.

In these various embodiments, the alpha-V-targeting immunoliposome includes an active entrapped in the liposomes, where ‘entrapped’ intends associated with the liposome lipid bilayer or with the internal aqueous compartments. The agent, in one embodiment, is a therapeutic agent, such as an antineoplastic agent. In a specific embodiment, the antineoplastic is a cytotoxic or cytostatic agent, such as doxorubicin.

In another embodiment, products prepared by the foregoing processes are disclosed. The products prepared include, but are not limited to a Fab′ preparation where heavy and light chains are minimized, a Fab′ immuno drug conjugate, a Fab′ immuno lipopolymer conjugate, and a Fab′ targeted liposome.

The following sections describe the antibodies that are used in the aforementioned processes and embodiments. The following sections also describe the liposome components that are used in some of the aforementioned processes and embodiments, including the liposome lipids and therapeutic agents, preparation of liposomes bearing an antibody, in some embodiments an anti-alpha-V targeting ligand, and methods of using the liposomal composition for treatment of disorders characterized by cellular expression of alpha-V-integrins such as αvβ3, αvβ5, and αvβ6 integrin receptors.

### A. Antibody

The antibodies that are prepared according to the processes set forth in this specification, are those that form heavy and light chains when subjected to reduction conditions. Generally these antibodies are those that have solvent accessible bonds that are broken during reduction conditions to the antibody leading to the formation of heavy and light chains. More specifically, the antibodies are those that have disulfide bridges between the heavy and light chains of the antibody that are broken during the reduction conditions that the antibody is exposed. In one embodiment, the antibody is one that does not selectively form Fab′ and produces heavy and light chains.

The aforementioned reduction conditions are those that are capable of reducing disulfide bonds, generally solvent accessible bonds, but do not impact adversely on the biological activity or binding affinity of the antibody. Reduction of the disulfide bonds between heavy and light chains in antibodies increases with increased reducing agent concentration, increased time periods of exposure of the antibody to the reducing agent, and higher temperatures and pHs. One of skill in the art, guided by the disclosure and examples set forth herein, would be able to identify if an antibody could be used in the processes disclosed in this specification.

In one embodiment, the antibody derived targeting ligand, as disclosed and defined herein, and prepared according to the processes set forth in this specification, may be derived from any anti-alpha-V specific antibody or selected from a library of preformed antibody-derived structures, e.g. a phage library comprising antibody Fab′ or scFv or Fv. In one embodiment, the antibody prepared in accordance with the processes set forth in this application, and in one embodiment used in the liposome composition described herein comprises antigen binding domains derived from a human anti-alpha-V antibody generated by immunization of a transgenic mouse containing genes for the expression of human immunoglobulins. Preparation of a parent anti-alpha-V antibody known as CNTO 95, from which the antigen binding domains are derived is described in Preparation of the antibody is described in detail in PCT publication no. WO 02/12501 and U.S. Pat. No. 7,163,681 both incorporated by reference herein.

The antibody-derived targeting ligand includes any protein or peptide containing molecule that comprises at least a portion of a complementarity determining region (CDR) of a heavy or light chain or a ligand binding portion thereof derived from the antibody designated “CNTO 95” (see PCT publication no. WO 02/12501 and U.S. Publication No. 2003/040044), in combination with a heavy chain or light chain variable region, a heavy chain or light chain constant region, a framework region, or any portion thereof, that can be incorporated into an antibody.

Preferably, the CDR1, 2, and/or 3 of the engineered targeting ligand described above comprise the exact amino acid sequence(s) as those of the fully human Mab designated CNTO 95, Gen0101, CNTO 95, C371A generated by immunization of a transgenic mouse as disclosed herein. However, the ordinarily skilled artisan will appreciate that some deviation from the exact CDR sequences of CNTO 95 may be possible while still retaining the ability of the antibody to bind alpha-V effectively (e.g., conservative substitutions). In a particular embodiment, the antibody or antigen-binding fragment can have an antigen-binding region that comprises at least a portion of at least one heavy chain CDR (i.e., CDR1, CDR2 and/or CDR3) having the amino acid sequence of the corresponding CDRs 1, 2 and/or 3 (as shown in SEQ ID NO: 1). In another particular embodiment, the antibody or antigen-binding portion or variant can have an antigen-binding region that comprises at least a portion of at least one light chain CDR (i.e., CDR1, CDR2 and/or CDR3) having the amino acid sequence of the corresponding CDRs 1, 2 and/or 3 (as shown in SEQ ID NO: 2) of the light chain of CNTO95. In a preferred embodiment the three heavy chain CDRs and the three light chain CDRs of the antibody or antigen-binding fragment have the amino acid sequence of the corresponding CDR of mAb CNTO 95 (as shown in SEQ ID Nos: 1 and 2). Accordingly, in another embodiment, the engineered antibody may be composed of one or more CDRs that are, for example, 90%, 95%, 98% or 99.5% identical to one or more CDRs of CNTO 95. Anti-alpha-V subunit antibodies can include, but are not limited to, at least one portion, sequence or combination selected from 5 to all of the contiguous amino acids of at least one of six CDRs shown in SEQ ID NOS: 1 and 2. An anti-alpha-V subunit antibody can further optionally comprise a polypeptide of at least one of 70-100% of the contiguous amino acids of at least one of SEQ ID NOS: 1 and 2. For example, the amino acid sequence of a light chain variable region can be compared with the sequence of SEQ ID NO: 2, residues 1-108, or the amino acid sequence of a heavy chain CDR3 can be compared with SEQ ID NO: 1, residues 1-119.

As disclosed and claimed herein, the sequences set forth in SEQ ID NOs. 1-4 include “conservative sequence modifications”, i.e. amino acid sequence modifications which do not significantly affect or alter the binding characteristics of the antibody encoded by the nucleotide sequence or containing the amino acid sequence. Such conservative sequence modifications include amino acid substitutions, additions and deletions. Modifications can be introduced into SEQ ID NOs: 1-2 or to the nucleic acids encoding them by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in a human anti-alpha-V antibody is preferably replaced with another amino acid residue from the same side chain family.

In another aspect, the structural features of a human anti-alpha-V antibody are used to create a structurally related human anti-alpha-V targeting ligand that retains the ability to bind to alpha-V. More specifically, one or more antigen binding regions, specifically the variable regions and the CDR regions of the anti-alpha-V antibody can be combined recombinantly with other known human constant regions or framework regions and CDRs to create additional, recombinantly-engineered, human anti-alpha-V targeting moieties that can be prepared in accordance with the processes disclosed herein. At least one antibody binds at least one specified epitope specific to at least one alphaV subunit protein, subunit, fragment, portion or any combination thereof. The at least one epitope can comprise at least one portion of said protein, preferably comprised of at least one extracellular, soluble, external or cytoplasmic portion of said protein. The at least one specified epitope can comprise any combination of at least one amino acid sequence of at least 1-3 amino acids to the entire specified portion of contiguous amino acids of a protein encoded by the ITGAV gene (Gene ID: 3683).

Amino acids in an anti-alpha-V antibody that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to at least one alpha-V subunit neutralizing activity. Sites that are critical for antibody binding can also be identified by structural analysis such as crystallization, nuclear magnetic resonance or photoaffinity labeling (Smith, et al., J. Mol. Biol. 224:899-904 (1992) and de Vos, et al., Science 255:306-312 (1992)).

The antibodies that are prepared according to the processes disclosed herein are not limited to CNTO 95 mAb, its variable domains, or CDR sequences. It is anticipated that any appropriate anti-alpha-V antibody and corresponding anti-αv CDRs described in the art may be substituted therefore. Other anti-αv subunit antibodies may be developed by screening hybridomas, combinatorial libraries, or specific antibody phage display libraries [W. D. Huse et al., 1988, Science, 246:1275-1281] for binding to a human αV-containing integrin epitope. A collection of antibodies, including hybridoma products or antibodies derived from any species immunoglobulin repertoire may be screened in a conventional competition assay, with one or more of the known anti-alpha-V antibodies described herein. Thus, antibodies, other than CNTO95 derived antibodies, may be provided for which are capable of binding to the αv-containing receptors.

In another embodiment, the anti-alpha-V antibody may be 17E6, a fragment, or variant thereof based on the binding domains of 17E6 as described in U.S. Pat. No. 5,985,278 which is incorporated herein by reference and which reacts with the αV-chain of human αV-integrins, blocking the attachment to the integrin substrate of the αV-integrin bearing cell, triggering reversal of established cell matrix interaction caused by αV-integrins, blocking tumor development, and showing no cytotoxic activity. In yet another embodiment, the anti-alpha-V antibody may be murine monoclonal B9 and the humanized antibody HuB9 as described in U.S. Pat. No. 6,160,099 which is incorporated herein by reference and which react with the αV-chain of human αᵥβ₃ and αᵥβ₅ integrins.

Variations derived from the naturally occurring antibody structure, as defined herein which are particularly useful include Fabs and scFv. ScFv (single-chain variable fragment antibody) is a minimal antibody moiety in which the variable regions from the heavy and light chains (Vh and Vl) of immunoglobulin are joined by a flexible linker (U.S. Pat. No. 5,260,203 which is incorporated herein by reference). The resulting linked domains represent a variable region fragment, which retains both affinity and specificity of the parent antibody. These small antibody fragments can be produced in E. coli providing a fast and economic manufacturing option. A scFv of CNTO95 was developed as a targeting moiety to specifically direct drug containing STEALTH liposomes to aVb3 and aVb5 integrins which are known to be present on numerous types of cancer cells as well as angiogenic endothelial cells thereby representing an ideal targeting opportunity for drug delivery to subjects with neoplastic disease. One particular advantage of the scFv is that, in contrast to larger antibody fragments, a scFv contains only 4 cysteine residues and these are engaged in the 2 disulfide bonds of the V-domains. This facilitates introduction of a free cysteine residue for chemical conjugation. Moreover, the small size of the scFv is less likely to impact the stability and low non-specific interactions of STEALTH Liposomes. A further advantage of an scFv with the alpha-V targeting properties of CNTO95 are the ability to cause receptor internalization upon binding. Certain specific embodiments of the anti-alpha-V targeting antibody constructs are single chain binding fragments (scFv) which may be prepared from a parent antibody as described in Example 11 and as exemplified by SEQ ID NO: 4.

In another embodiment the targeting antibody is a Fab, which represents a monovalent binding fragment of an antibody, comprising both heavy chain and light chain portions of an antibody, which may be produced by cleavage from an antibody or be synthesized recombinantly and expressed as the heterodimeric structure. Exemplary forms of Fabs produced by both processes are described in Example 2 and 9. A Fab derived from cleavage of the parent CNTO95 IgG comprising the full-length heavy and light chains of the antibody (SEQ ID NO: 1 and 2, respectively) cleaved by pepsin is represented by residues 1-234 or SEQ ID NO: 1 and the full-length light chain (SEQ ID NO: 2). A recombinantly engineered host cell line expressing and secreting a Fab (sFab) which is represented by SEQ ID NO: 3 and SEQ ID NO: 2 is particularly useful for the purposes of conjugation and insertion into a pre-formed liposome among other uses.

It is useful for the targeting antibody to comprise a predetermined site for conjugation to a chemically moiety capable of insertion into the lipid structure of the liposome. While chemical modification of and addition of reactive groups is possible by standard techniques, it is convenient to genetically encode such a site into the structure of the antibody whenever possible. In the case of the recombinantly expressed and secreted antibody targeting constructs, including the sFab and scFv antibody constructs, each polypeptide chain has an additional C-terminal tail amino acid sequence having a means for chemically modifying the polypeptide such as through a free sulfhydryl of a cysteine side chain or an amine residue of a lysine side chain. Exemplary methods of incorporating a predetermined site for conjugation are taught in e.g. U.S. Pat. No. 5,837,846 which is incorporated herein by reference and which embodiments include a C-terminal cysteine or a C-terminal tail peptide bonded to the C-terminus of the antibody heavy chain or heavy chain fragment or scFv and, optionally, having an amino acid sequence selected from the group consisting of Ser-Cys, (Gly)₄-Cys, and (His)₆-(Gly)₄-Cys thereby incorporating linking means as well as purification means (his-tag).

### B. Liposome Lipid Components

Liposomes suitable for use and employing the antibodies prepared in accordance with the processes described herein include those composed primarily of vesicle-forming lipids. Such a vesicle-forming lipid is one which can form spontaneously into bilayer vesicles in water, as exemplified by the phospholipids, with its hydrophobic moiety in contact with the interior, hydrophobic region of the bilayer membrane, and its head group moiety oriented toward the exterior, polar surface of the membrane. Lipids capable of stable incorporation into lipid bilayers, such as cholesterol and its various analogs, can also be used in the liposomes.

The vesicle-forming lipids are preferably lipids having two hydrocarbon chains, typically acyl chains, and a head group, either polar or nonpolar. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. The above-described lipids and phospholipids whose carbon chains have varying degrees of saturation can be obtained commercially or prepared according to published methods. Other suitable lipids include glycolipids, cerebrosides and sterols, such as cholesterol.

Cationic lipids are also suitable for use in the liposomes described herein, where the cationic lipid can be included as a minor component of the lipid composition or as a major or sole component. Such cationic lipids typically have a lipophilic moiety, such as a sterol, an acyl or diacyl chain, and where the lipid has an overall net positive charge. Preferably, the head group of the lipid carries the positive charge. Exemplary cationic lipids include 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethylammonium bromide (DORIE); N-[1-(2,3-dioleyloxy) propyl]-N,N,N-trimethylammonium chloride (DOTMA); 3 [N—(N′,N′-dimethylaminoethane)carbamoly] cholesterol (DC-Chol); and dimethyldioctadecylammonium (DDAB). The cationic vesicle-forming lipid may also be a neutral lipid, such as dioleoylphosphatidyl ethanolamine (DOPE) or an amphipathic lipid, such as a phospholipid, derivatized with a cationic lipid, such as polylysine or other polyamine lipids. For example, the neutral lipid (DOPE) can be derivatized with polylysine to form a cationic lipid.

The vesicle-forming lipid can be selected to achieve a specified degree of fluidity or rigidity, to control the stability of the liposome in serum, to control the conditions effective for insertion of the targeting conjugate, as will be described, and/or to control the rate of release of the entrapped agent in the liposome. Liposomes having a more rigid lipid bilayer, or a liquid crystalline bilayer, are achieved by incorporation of a relatively rigid lipid, e.g., a lipid having a relatively high phase transition temperature, e.g., up to 60° C. Rigid, i.e., saturated, lipids contribute to greater membrane rigidity in the lipid bilayer. Other lipid components, such as cholesterol, are also known to contribute to membrane rigidity in lipid bilayer structures.

On the other hand, lipid fluidity is achieved by incorporation of a relatively fluid lipid, typically one having a lipid phase with a relatively low liquid to liquid-crystalline phase transition temperature, e.g., at or below room temperature.

The liposomes also include a vesicle-forming lipid derivatized with a hydrophilic polymer. As has been described, for example in U.S. Pat. No. 5,013,556, including such a derivatized lipid in the liposome composition forms a surface coating of hydrophilic polymer chains around the liposome. The surface coating of hydrophilic polymer chains is effective to increase the in vivo blood circulation lifetime of the liposomes when compared to liposomes lacking such a coating.

Vesicle-forming lipids suitable for derivatization with a hydrophilic polymer include any of those lipids listed above, and, in particular phospholipids, such as distearoyl phosphatidylethanolamine (DSPE).

Hydrophilic polymers suitable for derivatization with a vesicle-forming lipid include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide and hydrophilic peptide sequences. The polymers may be employed as homopolymers or as block or random copolymers.

A preferred hydrophilic polymer chain is polyethyleneglycol (PEG), preferably as a PEG chain having a molecular weight between 500-10,000 daltons, more preferably between 750-10,000 daltons, still more preferably between 750-5000 daltons. Methoxy or ethoxy-capped analogues of PEG are also preferred hydrophilic polymers, commercially available in a variety of polymer sizes, e.g., 120-20,000 Daltons.

Preparation of Vesicle-Forming Lipids Derivatized with Hydrophilic Polymers has been described, for example in U.S. Pat. No. 5,395,619 which is incorporated herein by reference. Preparation of liposomes including such derivatized lipids has also been described, where typically between 1-20 mole percent of such a derivatized lipid is included in the liposome formulation (see, for example, U.S. Pat. No. 5,013,556 which is incorporated herein by reference).

### C. Preparation of Lipid-Polymer-Antibody Conjugate

The antibody, and in some embodiments the anti-alpha-V antibody, is covalently attached to the free distal end of a hydrophilic polymer chain, which is attached at its proximal end to a vesicle-forming lipid. There are a wide variety of techniques for attaching a selected hydrophilic polymer to a selected lipid and activating the free, unattached end of the polymer for reaction with a selected ligand, and in particular, the hydrophilic polymer polyethyleneglycol (PEG) has been widely studied (Allen, T. M., et al., Biochemicia et Biophysica Acta, 1237:99-108 (1995); Zalipsky, S., Bioconjugate Chem., 4(4):296-299 (1993); Zalipsky, S., et al. FEBS Lett., 353:71-74 (1994); Zalipsky, S. et al., Bioconjugate Chemistry, 6(6):705-708 (1995); Zalipsky, S., in STEALTH LIPOSOMES (D. Lasic and F. Martin, Eds.) Chapter 9, CRC Press, Boca Raton, Fla. (1995)).

Generally, the PEG chains are functionalized to contain reactive groups suitable for coupling with, for example, sulfhydryls, amino groups, and aldehydes or ketones (typically derived from mild oxidation of carbohydrate portions of an antibody) present in a wide variety of ligands. Examples of such PEG-terminal reactive groups include maleimide (for reaction with sulfhydryl groups), N-hydroxysuccinimide (NHS) or NHS-carbonate ester (for reaction with primary amines), hydrazide or hydrazine (for reaction with aldehydes or ketones), iodoacetyl (preferentially reactive with sulfhydryl groups) and dithiopyridine (thiol-reactive). Synthetic reaction schemes for activating PEG with such groups are set forth in U.S. Pat. Nos. 5,631,018, 5,527,528, 5,395,619, and the relevant sections describing synthetic reaction procedures are expressly incorporated herein by reference.

In supporting studies, the anti-integrin antibody fragment was a Fab′ antibody produced by enzymatic cleavage of a full length parent antibody, which was attached to a lipid-PEG conjugate, as described in Example 2. In brief, a lipopolymer with a reactive end, maleimide-PEG-DSPE, was inserted into drug loaded liposomes, for subsequence conjugation between the reactive PEG end and the Fab′ targeting ligand. The Fab′ was prepared by first reducing F(ab′)₂ to cleave solvent accessible disulfide bonds and then reoxidizing the protein in a controlled manner to selectively reform the disulfide bonds between the heavy and light chains, thus forming Fab′ at a high purity. The reoxidized Fab′ was then added to the liposomes bearing reactive maleimide groups to conjugate the Fab′ ligand to the external surface of the liposomes.

In another study, as described in Example 9, the anti-alpha-V antibody-derived construct was also a Fab′ fragment but was a variant of the parental sequence (SEQ ID NO: 3) synthesized by recombinant methods and conjugated to a PEGylated-lipid for surface insertion into a preformed liposome.

In another study, as described in Example 11, the anti-alpha-V antibody-derived construct was a scFv which was a produced variant of the parental sequence heavy chain (SEQ ID NO: 1) variable domain with the parental sequence light chain (SEQ ID NO: 2) variable domain with a flexible polypeptide linker interposed there between. The construct was produced by linking coding sequences for the variable domains operably with a coding sequence for the linking a sequence by recombinant methods. The expressed purified scFv, which retained binding specificity for alphav-integrins was conjugated to a PEGylated-lipid for surface insertion into a preformed liposome.

### D. Liposome Preparation

Various approaches have been described for preparing liposomes having a targeting ligand attached to the distal end of liposome-attached polymer chains. One approach involves preparation of lipid vesicles which include an end-functionalized lipid-polymer derivative; that is, a lipid-polymer conjugate where the free polymer end is reactive or “activated” (see, for example, U.S. Pat. Nos. 6,326,353 and 6,132,763 which are incorporated herein by reference). Such an activated conjugate is included in the liposome composition and the activated polymer ends are reacted with a targeting ligand after liposome formation. Example 2 describes preparation of liposomes using this approach.

In another approach, the lipid-polymer-ligand conjugate is included in the lipid composition at the time of liposome formation (see, for example, U.S. Pat. Nos. 6,224,903, 5,620,689 which are incorporated herein by reference).

In another method of preparing a targeted liposome, a micellar solution of the lipid-polymer-ligand conjugate is incubated with a suspension of liposomes and the lipid-polymer-ligand conjugate is inserted into the pre-formed liposomes (see, for example, U.S. Pat. Nos. 6,056,973 and 6,316,024 which are incorporated herein by reference). Examples 3, 9 and 11 describe preparation of liposomes using this approach.

It will be appreciated that liposomes carrying an entrapped agent and bearing surface-bound targeting ligands, i.e., targeted, therapeutic liposomes, are prepared by any of these approaches. A preferred method of preparation is the insertion method, where pre-formed liposomes are incubated with the targeting conjugate to achieve insertion of the targeting conjugate into the liposomal bilayers. In this approach, liposomes are prepared by a variety of techniques, such as those detailed in Szoka, F., Jr., et al., Ann. Rev. Biophys. Bioeng., 9:467 (1980), and specific examples of liposomes prepared in support of the inventions disclosed herein will be described below. Typically, the liposomes are multilamellar vesicles (MLVs), which can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium. The lipid film hydrates to form MLVs, typically with sizes between about 0.1 to 10 microns.

The liposomes can include a vesicle-forming lipid derivatized with a hydrophilic polymer to form a surface coating of hydrophilic polymer chains on the liposomes surface. Addition of a lipid-polymer conjugate is optional, since after the insertion step the liposomes will include lipid-polymer-targeting ligand. Additional polymer chains added to the lipid mixture at the time of liposome formation and in the form of a lipid-polymer conjugate result in polymer chains extending from both the inner and outer surfaces of the liposomal lipid bilayers. Addition of a lipid-polymer conjugate at the time of liposome formation is typically achieved by including between 1-20 mole percent of the polymer-derivatized lipid with the remaining liposome forming components, e.g., vesicle-forming lipids. Exemplary methods of preparing polymer-derivatized lipids and of forming polymer-coated liposomes have been described in U.S. Pat. Nos. 5,013,556, 5,631,018 and 5,395,619, which are incorporated herein by reference. It will be appreciated that the hydrophilic polymer may be stably coupled to the lipid, or coupled through an unstable linkage, which allows the coated liposomes to shed the coating of polymer chains as they circulate in the bloodstream or in response to a stimulus.

The liposomes also include a therapeutic or diagnostic agent, and exemplary agents are provided below. The selected agent is incorporated into liposomes by standard methods, including (i) passive entrapment of a water-soluble compound by hydrating a lipid film with an aqueous solution of the agent, (ii) passive entrapment of a lipophilic compound by hydrating a lipid film containing the agent, and (iii) loading an ionizable drug against an inside/outside or outside/inside liposome chemical or pH gradient. Other methods, such as reverse-phase evaporation, are also suitable.

After liposome formation, the liposomes can be sized to obtain a population of liposomes having a substantially homogeneous size range, typically between about 0.01 to 0.5 microns, more preferably between 0.03-0.40 microns. One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less (Martin, F. J., in SPECIALIZED DRUG DELIVERY SYSTEMS—MANUFACTURING AND PRODUCTION TECHNOLOGY, P. Tyle, Ed., Marcel Dekker, New York, pp. 267-316 (1990)).

In one embodiment, after formation of the liposomes, a targeting ligand is incorporated to achieve a target-cell sensitized, therapeutic liposome. The targeting ligand can be incorporated by attaching the ligand to an activated end on the hydrophilic polymer chain (Example 2) or by incubating the pre-formed liposomes with the lipid-polymer-ligand conjugate (Examples 3, 9, and 11). In the latter approach, the pre-formed liposomes and the conjugate are incubated under conditions effective to achieve insertion of the conjugate into the liposome bilayer. More specifically, the two components are incubated together under conditions which achieve insertion of the conjugate in such a way that the targeting ligand is oriented outwardly from the liposome surface, and therefore available for interaction with its cognate receptor. It will be appreciated that the conditions effective to achieve insertion of the targeting conjugate into the liposome are determined based on several variables, including, the desired rate of insertion, where a higher incubation temperature may achieve a faster rate of insertion, the temperature to which the ligand can be safely heated without affecting its activity, and to a lesser degree the phase transition temperature of the lipids and the lipid composition. It will also be appreciated that insertion can be varied by the presence of solvents, such as amphipathic solvents including polyethyleneglycol and ethanol, or detergents.

The targeting conjugate, in the form of a lipid-polymer-ligand conjugate, will typically form a solution of micelles when the conjugate is mixed with an aqueous solvent. The micellar solution of the conjugates is mixed with a suspension of pre-formed liposomes for insertion of the conjugate into the liposomal lipid bilayers. Accordingly, in another aspect, a plurality of targeting conjugates, such as a micellar solution of targeting conjugates, for use in preparing a targeted, therapeutic liposome composition, is contemplated. Each conjugate is composed of (i) a lipid having a polar head group and a hydrophobic tail, (ii) a hydrophilic polymer having a proximal end and a distal end, where the polymer is attached at its proximal end to the head group of the lipid, and (iii) an anti-alpha-V antibody targeting ligand attached to the distal end of the polymer.

### Exemplary Immunoliposomes

In supporting studies, immunoliposomes having an anti-αv integrin Fab antibody were prepared as described in Example 1 and 2 and with an alternative embodiment of a Fab secreted by an engineered host cell, in Example 9. In another embodiment, immunoliposomes having an anti-αv integrin scFv targeting moiety were prepared as described in Example 12. In brief, liposomes were prepared from the lipids HSPC, cholesterol. The therapeutic agent doxorubicin was loaded into the liposomes by remote loading against an ammonium ion gradient (Doxil®). In one method of attaching the targeting moiety to the liposome, an anti-αV Fab having a free sulhydryl near the C-terminus was attached to the active end of the PEG chains previously inserted as Mal-PEG-DSPE. Liposome formulations having various antibody:liposome ratios were prepared. In an alternate method of attaching an anti-αV Fab, a Fab having a free sulhydryl near the C-terminus can be conjugated to the Mal-PEG-DSPE and the Fab-PEG-DSPE conjugate inserted into pre-formed liposomes as taught in Example 3 and Example 9. While Example 12 is directed to a scFv-PEG-DSPE that is conjugated to a post MaIPEG-DSPE inserted liposome, other scAb-PEG-lipids exist that do not denature in insertion conditions and may also be inserted into pre-formed, preloaded liposomes at various ligand to liposome ratios.

In certain embodiments the alpha-V-targeted liposomes described in the examples set forth below were characterized, in vitro and in certain examples, in vivo.

### III. Methods of Use

The liposomes prepared from the antibodies prepared in accordance with the processes described herein can include a therapeutic or diagnostic agent in entrapped form. Entrapped is intended to include encapsulation of an agent in the aqueous core and aqueous spaces of liposomes as well as entrapment of an agent in the lipid bilayer(s) of the liposomes. Agents contemplated for use in the composition of the invention are widely varied, and examples of agents suitable for therapeutic and diagnostic applications are given below.

The targeting ligand included in the liposomes serves to direct the liposomes to a region, tissue, or cell bearing αvβ3, αvβ5 integrin, or other αv-subunit containing integrin receptors. Targeting the liposomes to such a region achieves site specific delivery of the entrapped agent. Disease states having a strong αvβ3, αvβ5 vascular disorders or osteoporosis (αvβ3); tumor angiogenesis, tumor metastasis, tumor growth, multiple sclerosis, neurological disorders, asthma, vascular injury or diabetic retinopathy (αvβ3 or αvβ5); and, angiogenesis (both αvβ3 and αvβ5).

Additionally, αvβ3 inhibitors or agents which block ligand binding to the receptor have been found to be useful in treating diseases characterized by excessive or inappropriate angiogenesis (i.e. formation of new blood vessels) and inhibiting neoplastic growth and tumor metastasis. Consequently the delivery of an appropriate therapeutic agent to would be expected to enhance this effect.

Moreover, the growth of tumors depends on an adequate blood supply, which in turn is dependent on the growth of new vessels into the tumor; thus, inhibition of angiogenesis can cause tumor regression in animal models (Harrison's Principles of Internal Medicine, 1991, 12th ed.). Therefore, an αv-subunit containing integrin-targeted liposome containing a therapeutic agent, which inhibit angiogenesis can be useful in the treatment of cancer by inhibiting tumor growth (Brooks et al., Cell, 79:1157-1164 (1994)). Evidence has also been presented suggesting that angiogenesis is a central factor in the initiation and persistence of arthritic disease and that the vascular integrin αvβ3 may be a preferred target in inflammatory arthritis. Therefore, αvβ3 targeted liposomes that deliver an anti-angiogenesis or appropriate therapeutic drug to treat arthritis may represent a novel therapeutic approach to the treatment of arthritic disease, such as rheumatoid arthritis (C. M. Storgard et al., J. Clin. Invest., 103:47-54 (1999)).

Inhibition of the αvβ5 integrin receptor can also prevent neovascularization. A monoclonal antibody for αvβ5 has been shown to inhibit VEGF-induced angiogenesis in rabbit cornea and the chick chorioallantoic membrane model (M. C. Friedlander et al., Science, 270:1500-1502 (1995)). Thus, anti-alpha-V targeted liposomes, which will naturally target αvβ5, containing an appropriate therapeutic agent would be useful for treating and preventing macular degeneration, diabetic retinopathy, cancer, and metastatic tumor growth.

Inhibition of αβ integrin receptors can also prevent angiogenesis and inflammation by acting as antagonists of alpha-V-subunit integrins comprising other β subunits, such as αvβ6 and αvβ8 (Melpo Christofidou-Solomidou et al., American Journal of Pathology, 151:975-83 (1997); Xiao-Zhu Huang et al., Journal of Cell Biology, 133:921-28 (1996)), again suggesting in disease states where angiogenesis or inflammation is to be treated that αvβ6 targeted liposome containing an appropriate therapeutic agent would provide a novel therapy.

More generally, the anti-alpha-V subunit antibodies or specified variants thereof can be used to measure or effect in an cell, tissue, organ or animal (including mammals and humans), to diagnose, monitor, modulate, treat, alleviate, help prevent the incidence of, or reduce the symptoms of, at least one condition mediated, affected or modulated by alpha-V integrins. Such conditions are selected from, but not limited to, diseases or conditions mediated by cell adhesion and/or angiogenesis. Such diseases or conditions include an immune disorder or disease, a cardiovascular disorder or disease, an infectious, malignant, and/or neurologic disorder or disease, or other known or specified alpha-V integrin subunit related conditions. In particular, the antibodies are useful for the treatment of diseases that involve angiogenesis such as disease of the eye and neoplastic disease, tissue remodeling such as restenosis, and proliferation of certain cells types particularly epithelial and squamous cell carcinomas. Particular indications include use in the treatment of atherosclerosis, restenosis, cancer metastasis, rheumatoid arthritis, diabetic retinopathy and macular degeneration. The neutralizing antibodies of the invention are also useful to prevent or treat unwanted bone resorption or degradation, for example as found in osteoporosis or resulting from PTHrP overexpression by some tumors. The antibodies may also be useful in the treatment of various fibrotic diseases such as idiopathic pulmonary fibrosis, diabetic nephropathy, hepatitis, and cirrhosis.

Thus, in one embodiment, a method is provided for modulating or treating at least one alpha-V subunit related disease, in a cell, tissue, organ, animal, or patient, as known in the art or as described herein, using at least one alpha-V subunit antibody of the present invention. One preferred indication is a malignant disease in a cell, tissue, organ, animal or patient. Malignant diseases include, but are not limited to, at least one of: leukemia, acute leukemia, acute lymphoblastic leukemia (ALL), B-cell, T-cell or FAB ALL, acute myeloid leukemia (AML), chromic myelocytic leukemia (CML), chronic lymphocytic leukemia (CLL), hairy cell leukemia, myelodyplastic syndrome (MDS), a lymphoma, Hodgkin's disease, a malignant lymphoma, non-hodgkin's lymphoma, Burkitt's lymphoma, multiple myeloma, Kaposi's sarcoma, colorectal carcinoma, pancreatic carcinoma, renal cell carcinoma, breast cancer, nasopharyngeal carcinoma, malignant histiocytosis, paraneoplastic syndrome/hypercalcemia of malignancy, solid tumors, adenocarcinomas, squamous cell carcinomas, sarcomas, malignant melanoma, particularly metastatic melanoma, hemangioma, metastatic disease, cancer related bone resorption, cancer related bone pain, and the like.

The immunoliposome includes an agent entrapped within the liposome. The agent is entrapped in either or both of the aqueous spaces and/or the lipid bilayers. The agent is an active, typically a therapeutic agent, which includes natural and synthetic compounds having the following therapeutic activities including but not limited to: steroids, immunosuppressants, antihistamines, non-steroidal anti-asthmatics, non-steroidal anti-inflammatory agents, cyclooxygenase-2 inhibitors, cytotoxic agents, gene therapy agents, radiotherapy agents, and agents capable of gene knockdown. Imaging agents may also be used in the targeted liposomes particularly with regard to diagnosis or imaging of patients who have cells and tissues sensitized to alpha-V-targeted liposomes.

Examples of these compounds include (a) steroids such as beclomethasone, methylprednisolone, betamethasone, prednisone, dexamethasone, and hydrocortisone; (b) immunosuppressants such as FK-506 type immunosuppressants; (c) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (d) non-steroidal anti-asthmatics such as beta2-agonists (terbutaline, metaproterenol, fenoterol, isoetharine, albuterol, bitolterol, salmeterol and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (e) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (f) cyclooxygenase-2 (COX-2) inhibitors such as celecoxib, rofecoxib, and parecoxib; (g) cholesterol lowering agents such as HMG-CoA reductase inhibitors (lovastatin, simvastatin, pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (h) anti-diabetic agents such as insulin, sulfonylureas, biguanides (metformin), a-glucosidase inhibitors (acarbose) and glitazones (troglitazone, pioglitazone, englitazone, MCC-555, BRL49653 and the like); (1) agents that interfere with TNF such as antibodies to TNF (REMICADE®) or soluble TNF receptor (e.g. ENBREL®); (h) anticholinergic agents such as muscarinic antagonists (ipratropium nad tiatropium); (i) antimetabolites such as azathioprine and 6-mercaptopurine, and cytotoxic cancer chemotherapeutic agents.

The entrapped therapeutic agent is, in one embodiment, a cytotoxic drug. The drug can be an anthracycline antibiotic, including but not limited to doxorubicin, daunorubicin, epirubicin, and idarubicin, including salts and analogs thereof. The cytotoxic agent can also be a platinum compound, such as cisplatin, carboplatin, ormaplatin, oxaliplatin, zeniplatin, enloplatin, lobaplatin, spiroplatin, ((−)-(R)-2-aminomethylpyrrolidine (1,1-cyclobutane dicarboxylato)platinum), (SP-4-3(R)-1,1-cyclobutane-dicarboxylato(2-)-(2-methyl-1,4-butanediamine-N,N′)platinum), nedaplatin and (bis-acetato-ammine-dichloro-cyclohexylamine-platinum(IV)). The cytotoxic agent can also be a topoisomerase 1 inhibitor, including but not limited to topotecan, irinotecan, (7-(4-methylpiperazino-methylene)-10,11-ethylenedioxy-20(S)-camptothecin), 7-(2-(N-isopropylamino)ethyl)-(20S)-camptothecin, 9-aminocamptothecin and 9-nitrocamptothecin. The cytotoxic agent can also be a vinca alkaloid such as vincristine, vinblastine, vinleurosine, vinrodisine, vinorelbine, and vindesine. The entrapped therapeutic agent can also be an angiogenesis inhibitor, such as angiostatin, endostatin and TNF.

Nucleic acids are also contemplated for use as the therapeutic agent. DNA and RNA based nucleic acids, including fragments and analogues, can be used for treatment of various conditions, and coding sequences for specific genes of interest can be retrieved from DNA sequence databanks, such as GenBank or EMBL. For example, polynucleotides for treatment of viral, malignant and inflammatory diseases and conditions, such as, cystic fibrosis, adenosine deaminase deficiency and AIDS, have been described. Treatment of cancers by administration of tumor suppressor genes, such as APC, DPC4, NF-1, NF-2, MTS1, RB, p53, WT1, BRCA1, BRCA2 and VHL, are contemplated. Administration of the following nucleic acids for treatment of the indicated conditions are also contemplated: HLA-B7, tumors, colorectal carcinoma, melanoma; IL-2, cancers, especially breast cancer, lung cancer, and tumors; IL-4, cancer; TNF, cancer; IGF-1 antisense, brain tumors; IFN, neuroblastoma; GM-CSF, renal cell carcinoma; MDR-1, cancer, especially advanced cancer, breast and ovarian cancers; and HSV thymidine kinase, brain tumors, head and neck tumors, mesothelioma, ovarian cancer.

The polynucleotide can be an antisense DNA oligonucleotide composed of sequences complementary to its target, usually a messenger RNA (mRNA) or an mRNA precursor. The mRNA contains genetic information in the functional, or sense, orientation and binding of the antisense oligonucleotide inactivates the intended mRNA and prevents its translation into protein. Such antisense molecules are determined based on biochemical experiments showing that proteins are translated from specific RNAs and once the sequence of the RNA is known, an antisense molecule that will bind to it through complementary Watson-Crick base pairs can be designed. Such antisense molecules typically contain between 10-30 base pairs, more preferably between 10-25, and most preferably between 15-20. The antisense oligonucleotide can be modified for improved resistance to nuclease hydrolysis, and such analogues include phosphorothioate, methylphosphonate, phosphodiester and p-ethoxy oligonucleotides (WO 97/07784). The entrapped agent can also be a ribozyme or catalytic RNA.

Typically, treatment of pathologic conditions is effected by administering an effective amount or dosage of an anti-alpha-V subunit antibody immunoliposome composition. In some patients and for some conditions, the anti-alpha-V antibody has a therapeutic activity, and in these situations the amount of antibody administered can range, on average, from at least about 0.01 to 500 milligrams of at least one anti-alpha-V subunit antibody per kilogram of patient per dose, and preferably from at least about 0.1 to 100 milligrams antibody/kilogram of patient per single or multiple administration, depending upon the specific activity of contained in the composition. Alternatively, the effective serum concentration can comprise 0.1-5000 μg/mL serum concentration per single or multiple administration. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

For other patients and for other diseases, the anti-alpha-V antibody serves as a targeting ligand, to direct the liposome and its entrapped therapeutic drug to a specific site in vivo. In these cases, the dosage of immunoliposome is selected according to the desired serum concentration of the entrapped therapeutic drug.

For other patients and for other diseases, the anti-alpha-V antibody has a therapeutic effect and the entrapped drug has a therapeutic effect. The dosage of the immunoliposome composition will then be selected according to the desired serum concentration of the drug and/or the antibody, as can be determined from in vitro cytotoxicity tests and/or in vivo dosing studies.

The dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. The dosage can be a one-time or a periodic dosage given at a selected interval of hours, days, or weeks.

Any route of administration is suitable, with intravenous and other parenteral modes being preferred.

In another aspect, a combined treatment regimen is contemplated, where the immunoliposome composition described above is administered in combination with a second agent. The second agent can be any therapeutic agent, including other drug compounds as well as biological agents, such as peptides, antibodies, and the like. The second agent can be administered simultaneously with or sequential to administration of the immunoliposomes, by the same or a different route of administration.

### IV. Examples

The following examples are illustrative in nature and are in no way intended to be limiting.

### Materials

Hydrogenated soy phosphatidylcholine (HSPC) was purchased from Lipoid K.G. (Ludwigshafen, Germany). Cholesterol was received from Croda, Inc. (New York, N.Y.) and N-(carbonyl-methoxypolyethylene glycol 2000)-1,2-distearoyl-sn-glycero-3-phosphatidylethanolamine, sodium salt (mPEG-DSPE) was received from Genzyme (Cambridge, Mass.). Doxorubicin hydrochloride was received from Meiji Seika Kaisha Ltd. (Tokyo, Japan).

Dithioerythritol (DTE), ethylenediaminetetraacetic acid (EDTA), iodoacetamide (IAC), N-ethylmaleimide (NEM), sodium phosphate monobasic, sodium phosphate dibasic, NaCl, and copper (II) chloride dihydrate were purchased from Sigma (St. Louis, Mo.). Maleimide-terminated PEG coupled to DSPE (MaIPEG-DSPE) was purchased from Avanti Polar Lipids (Alabaster, Ala.). The desalting columns, HiTrap SP HP ion exchange columns, and the sephacryl 300 size-exclusion columns were purchased from Amersham Biosciences (Piscataway, N.J.).

### Example 1

### Preparation of Liposomes

### 1. Liposome Preparation

Liposome-entrapped doxorubicin was prepared using methods previously described (e.g., U.S. Pat. No. 5,013,556). In brief, the lipid components (HSPC, CHOL, mPEG-DSPE at a molar ratio of 56.4:38.3:5.3) were solubilized in ethanol and added to 250 mM ammonium sulfate solution at 60-65° C. The solution was mixed for 1 hour at this elevated temperature to allow for hydration of the lipid components and formation of liposomes. The liposomes were downsized below a mean particle size of 100 nm by extrusion. The process fluid was diafiltered with ammonium sulfate solution to remove the ethanol, followed by sucrose solution to remove the ammonium sulfate in the external liposomal phase. A sample of the post diafiltration process fluid was submitted for phosphorus concentration determination and diluted to a target phosphorus concentration based on the measured value. Doxorubicin was loaded into the liposomes by incubating the liposomal process fluid with doxorubicin drug solution at 60-65° C. for 1 hour. The resulting drug loaded liposomes were cooled and stored at 2-8° C.

### Example 2

### Preparation of an Anti-Alpha-V Fab Using a Protease

### 1. Parent Antibody

The isolated parent antibody, CNTO 95, a heterodimer consisting of SEQ ID NO: 1 and SEQ ID NO: 2 as disclosed in U.S. Pat. No. 7,163,681; was desired as the source of Fab′ used as a targeting-ligand. CNTO95 is a full-length human antibody of the IgG1k type. The monovalent binding arm, Fab′, to be used represents residues 1-234 or the heavy chain (SEQ ID NO: 1) and the entire light chain (SEQ ID NO: 2).

### 2. Preparation of F(ab′)2

Cleavage of CNTO95 with pepsin under conditions to release the Fc portion from the (Fab′)₂ of the antibody was performed. Starting with CNT095 purified using Protein A chromotography, the antibody was diafiltered into 0.1 M Citrate pH 4.2 to a final concentration of 10 g/L. Pepsin (Sigma Cat no P6887), reconstituted as a stock solution in the same buffer, was added at a final concentration of 100 U Enzyme/mg IgG and allowed to digest for 90 min at 40° C. The digestion was stopped by raising the pH to 6.0 with Tris-base and the material was filtered using a 0.22 um cut-off membrane.

CNTO95 F(ab′)2 proved to have some affinity for protein A column and therefore, to improve the yield, the pepsin digest was first purified using cation exchange chromatography Sepharose HP (GE Healthcare, Piscataway N.J., Cat. No. 17-1087-01) prior to it being passed over Protein A conjugated beads (MABSELECT™, GE Healthcare, Piscataway N.J., Cat. No. 17-5199-01) in a flowthrough mode.

The protein was further purified by anion exchange using a Q Sepharose™ XL (GE Healthcare, Cat No. 17-5072-01) in a flowthrough mode. The product is final purified by ultrafiltration using a 30 kDa MW cut-off membrane and finally concentrated to 10 mg/mL with 30 mM Na₂HPO₄ pH 6.0.

### 3. Reduction of F(ab′)₂

F(ab′)₂ was diluted with saline to a target protein concentration of 3.5 mg/mL. The pH of the protein solution was adjusted to 6.5 using 1 M sodium phosphate monobasic and 1M sodium phosphate dibasic. A 150 mM dithioerythritol (DTE) stock solution was prepared by dissolving the DTE in the correct volume of water. The volume of 150 mM DTE solution to achieve a 13 mM concentration when added to the protein solution was calculated. The protein was placed in a water bath set to 40° C. Sufficient time was allowed for the protein solution to reach 40° C. prior to adding the reducing agent. The correct volume of DTE was added to the protein solution and incubated at 40° C. for 60 minutes while mixing. At the end of the incubation time the protein solution was placed on ice.

DTE was removed by passing the protein solution over a desalting column. The column was prepacked with Sephadex G-25 with a diameter and height of 2.6 and 10 cm respectively. Up to 20 ml of solution could be loaded on the column for separation of protein from reducing agent. For volumes greater than 20 mL, the desalting step was done in batches. The running buffer used was 30 mM sodium phosphate buffer, pH 6.0 that was argon sparged. The low salt concentration of the running buffer allowed for efficient binding of the protein to the ion exchange column in the next step. As a note, ultra pure water (Milli Q system) was used in making all solutions and buffers to minimize any potential contamination of heavy metals that could affect the reoxidation rate. The flow rate over the column was 10 mL/min.

### 4. Ion Exchange Step

The protein solution was next loaded onto a HiTrap SP HP ion exchange column The column size was based on loading approximately 10 mg of protein per 1 mL of column packing. The flow rate during the loading step was ½ a column volume per minute. After loading all of the protein, the column was washed with 10 column volumes of 30 mM sodium phosphate buffer, pH 6.0 that was argon sparged in order to remove any residual DTE. Next, the column was washed with 10 column volumes of 30 mM sodium phosphate buffer, pH 6.0 that was air sparged. The protein was eluted from the column with 30 mM sodium phosphate buffer, 60 mM NaCl, pH 6.0 that was air sparged. The purpose for sparging the buffers in air at room temperature was to saturate the buffers with oxygen and make the process reproducible.

The pH of the eluted protein solution was checked and adjusted if necessary to 6.0. The protein concentration of the protein was determined and diluted to a value of 1.02 mg/mL with the same buffer used to elute the protein (30 mM sodium phosphate buffer, 60 mM NaCl, pH 6.0 that was air sparged). The protein solution was placed in a glass container with the appropriate capacity to minimize the headspace in the container and placed in a water bath set to 20° C.

### 5. Reoxidation Process

A 63.75 μM CuCl² stock solution was prepared. This low concentration was achieved by first making a 15.94 mM stock solution by dissolving the appropriate amount of CuCl² in water and performing successive dilutions in water until the final concentration was achieved. 20 μL of the 63.75 μM CuCl² stock solution was added for every 1 mL of protein solution. After mixing, the protein concentration was 1.00 mg/mL and the CuCl² concentration was 1.25 μM. It should be understood that while the introduction of trace amounts of Cu⁺² promotes oxidation of heavy and lights chains to Fab, other materials known to those of skill in the art to promote oxidation of heavy and light chains to Fab can be used, such as Fe⁺², Co+² and Mn⁺². While 1.25 μM of CuCl+² was used, the concentration could be in the range from about 0 to 12.5 mm/ml. Also, other protein concentrations were successfully employed such as 0.5, 0.75, and 0.85 mg/mL. The temperature during the reoxidation process was in the range from about 10 to about 20° C., with the reoxidation reaction rate increasing with increasing temperature. The time for the reoxidation reaction was about 4 to about 6 hours.

Samples were taken throughout the reoxidation process to monitor the extent of the reoxidation. The samples were run on an HPLC system with a size exclusion column and a running buffer containing SDS. The Fab′ peak was resolved from the heavy and light chain peaks allowing for the quantitation of the % Fab′ at the time the sample was taken. Based on these results, the time for reoxidation was determined. The time course for the reoxidation process was nearly identical for all batches made with an optimal time for reoxidation of 320 minutes. While a size exclusion chromatorgraphy was employed, it can be understood that the use of other purification approaches could be used such as diafiltration.

6. Maleimide-Terminated PEG Conjugated to DSPE Insertion into Preformed Liposomes

MaIPEG-DSPE was dissolved in water for injection at a concentration of 10 mg/mL. The volume of MaIPEG-DSPE solution to add to the liposomal solution was calculated based on 1) the phosphorus concentration of the post drug loaded liposomes, 2) the assumption that each liposome is comprised of 80,000 phospholipids and 3) 800 MaIPEG-DSPE molecules are inserted per liposome. The calculated amount of MaIPEG-DSPE solution was then added to the appropriate amount of post drug loaded liposomal solution, prepared as in Example 1, and incubated at 60 to 65° C. for 1 hour followed by cooling in an ice bath. 9% NaCl solution was added to the process fluid at a volume ratio of 1 to 9 to bring the solution up to 0.9% NaCl concentration. Addition of salt was deemed necessary to minimize any potential Fab′ denaturation under low salt conditions during the conjugation step. The solution pH was adjusted to 6.0 using either 1M sodium phosphate monobasic or 1M sodium phosphate dibasic. The preparation of the inserted liposomal material was typically completed a couple of hours prior to the conjugation step to minimize any potential inactivation of MaIPEG-DSPE over time.

### 7. Conjugation Step

At the end of reoxidation, the appropriate volume of protein solution was added to the post MaIPEG-DSPE inserted liposomes to begin the conjugation process. The amount of protein required was calculated based on 1) the desired Fab to liposome ratio, 2) the assumption that each liposome is comprised of 80000 phospholipid molecules, 3) phosphorus concentration of the post inserted solution and 4) the assumption that 50% of the protein in solution will conjugate as Fab. The last assumption was based on small-scale optimization work. For some of the later batches produced, EDTA solution was added to the liposomal and protein solutions to achieve a 1 mM concentration in the final mixture. This addition minimized any potential reoxidation during the conjugation process. Conjugation was at room temperature for 2 hours followed by overnight storage at 2-8° C. While EDTA was used, it should be understood that there are other chelating agents that are known to those of skill in the art that would be sufficient to minimize any potential reoxidation during the conjugation process.

### 8. Quenching and Final Column Purification

The conjugated liposomal formulations were quenched at a 1 mM cysteine concentration for 10 minutes prior to loading on the size exclusion column. The column contained sephacryl-300 packing with a diameter/height of 1.6/60 or 2.6/60 cm depending on the volume of solution to load on the column. A large volume (20% of the column volume) could be loaded on the column due to the large size difference between the liposomes and the unconjugated protein. The column removed unconjugated protein, unreacted cysteine and unencapsulated doxorubicin. The running buffer was 10 mM histine in saline, pH 6.5. The liposomal fraction was concentrated to a target drug concentration of 2.0 mg/mL with a centri prep concentrator with a 100K MWCO membrane at 2800 rpm.

The final formulations were submitted for potency, % drug encapsulation, particle size, pH, % Fab insertion and endotoxin. The reoxidation process was evaluated through analysis of both blocked and conjugated samples taken throughout the process. The samples were analyzed by SDS gel electrophoresis and the bands were quantified by densitometry measurements. Tables 1A-1B below summarize the characteristics of two batches of liposomes.

TABLE 1A
Characteristics of targeted liposomes in Batch 1

| Batch 1: Targeting | | | Particle | |
|---|---|---|---|---|
| ligand:liposome | Potency | % drug | Size | |
| ratio | (mg/mL) | encapsulation | (nm) | % insertion |
| 15:1 | 2.21 | 99 | 85 | 98.7 |
| 40:1 | 2.19 | 99 | 87 | 98.8 |
| 90:1 | 2.09 | 97 | 88 | 99.7 |

TABLE 1B
Characteristics of targeted liposomes in Batch 2

| Batch 2: Targeting | | | Particle | |
|---|---|---|---|---|
| ligand:liposome | Potency | % drug | Size | |
| ratio | (mg/mL) | encapsulation | (nm) | % insertion |
| 15:1 | 2.15 | 99 | 85 | 98.4 |
| 40:1 | 2.12 | 98 | 87 | 98.5 |
| 90:1 | 2.24 | 98 | 90 | 99.5 |

### Example 3

### In Vitro Binding and Internalization

This study evaluates the ability of integrin-targeted liposomes to achieve ligand mediated specific binding, internalization, and cell cytotoxicity in tumor cells bearing a humanized αVβ3/5 integrin receptor, as compared to liposomes lacking a targeting ligand.

### 1. Cells and Cell Media

Several human tumor cells bearing human αVβ3/5 integrins were used: (1) A375.S2, human melanoma cell line; (2) MDA-MB-231, Human Breast Carcinoma Cell line; (3) A2780, Human Ovarian Carcinoma Cell line; (4) HT29, Human Colon Carcinoma Cell line; (5) A549, Human Lung Carcinoma Cell line. As CNTO95 does not bind murine αVβ3/5 integrin, a murine melanoma cell line, B16F10, was used as a negative control in the study.

The media for each cell line was as follows:
1. A375.S2 cell, MEM (Minimum Essential Medium, ATCC Cat No. 30-2006) with addition of 10% Fetal Bovine Serum (FBS, ATCC, Cat No. 30-2021).
2. MDA-MB-231 cell, Leibovitz's L-15 Medium, (ATCC Cat No. 30-2008) with addition of 10% FBS.
3. A2780 cell, RPMI Medium 1640 (Gibco, Cat No. 22400-089HT29) with addition of 10% FBS.
4. A549 cell, F-12K Medium (ATCC, Cat No. 30-2004) with addition of 10% FBS.
5. B16-F10 cell, DMEM (Dulbecco's Modified Eagles's Medium, ATCC Cat No. 30-2002), with addition of 10% FBS.

Cell viability was assayed using the CellTiter 96 AQueous One Solution Cell Proliferation Assay from Promega (Cat No. G3581). A Spectra Max 250 plate reader was used, with a reading wavelength of 490 nm. Confocal Microscopy was done using a Nikon, Eelipse, E600. An Eppendorf centrifuge 5804 was used.

### 2. Liposome Compositions

Liposomes were prepared as described in Example 1, except in two aspects. First, Dextran Alexa Fluor 488 (Cat No. D-22910, from Molecular Probes) was included in the hydration buffer during the passive encapsulation step of liposome formation, and after the sizing step dialysis was used to remove any unencapsulated Alexa. Second, instead of inserting Fab-PEG-lipid into the liposome bilayer, maleimide-PEG-lipid (MaIPEG-DSPE) was inserted into the liposome bilayer at approximately 800 MalPeg-DSPE per liposome at 60° C. for 1 Hr. The insertion step was followed by the conjugation of the Fab to the reactive end of the lipopolymer. The appropriate amount of Fab was added to the MaIPEG-DSPE inserted liposomes to achieve a 90:1 anti-alpha-V-Fab targeting ligands per liposome ratio. These liposomes were used in the binding and internalization studies described below.

Liposomes bearing 15:1, 40:1, 90:1 and 180:1 alpha-integrin Fab targeting ligands per liposome and containing doxorubicin were prepared as described in Example 1 and 2. These targeted-liposomes were used in the cytotoxicity assay using the various cells lines, as described below.

### 3. Binding and Internalization Studies

A375.S2 cells were harvested by scraping and then resuspended to obtain individualized cells and rejuvenated for 1 hour, at 37° C. About 1 million cells of each tumor type were counted and distributed into individual centrifugation tubes. The tubes were spun to obtain a cell pellet.

For binding only studies, the cells were cooled to 4° C. by immersing the cell tubes in ice for 10 minutes and then treating with the targeted liposome composition containing a fluorescent marker (Dextran Alexa Fluor 488) at 4° C. for 30 minutes, with mild shaking (140 rpm). After the 30 minute incubation period, 1 mL of cold serum free media was added, the mixture was vortexed briefly, and the centrifuged. The cell pellet was resuspended with cold serum free media, shaken vigorously (440 rpm) at 4° C. for 10 minutes, and then centrifuged to recover the cell pellet. The cell pellet was left in about 100 μL of cold media and about 8 μL was taken for observation under a confocal microscope. All steps, except observation under the confocal microscope, were conducted at 4° C.

For binding and internalization, the cells were treated with the targeted liposome formulation at 37° C. for 10 min with mild shaking (140 rpm). Cells were treated with the liposome formulations containing either a doxorubicin payload or a fluorescent marker (Dextran Alexa Fluor 488). Treatment was terminated by adding 1 mL of washing media (serum free), vortexing briefly, and centrifuging to recover a cell pellet The cells in the pellet were resuspend in washing media, vigorously shaken for 10 minutes at 37° C. and then centrifuged again (440 rpm). The cell pellet was left in 100 μL of media, an aliquot of 8 μL was taken and placed on a glass slide for observation under a confocal microscope.

Binding and internalization of the targeted liposomes to A375.S2 cells was evaluated after various incubation times of the cells and the liposome formulation. Results are shown in FIGS. 1-4.

In FIGS. 1A and 1C, confocal microscopy results show that the targeted liposome formulation containing a fluorescent marker (Dextran Alexa Fluor 488) binds specifically to A375.S2 cells at 4° C. in vitro while the corresponding untargeted liposome formulation containing fluorescent marker does not. FIGS. 1B and 1D show images of the cells in “differential interference contrast” mode (DIC) and provides a reference on cell locations for FIGS. 1A and 1C. All subsequent Figures have a DIC pictures that correspond to the confocal image for reference. Confocal microscopy results shown in FIGS. 2A through 2H that liposomes bearing 90:1 alpha-integrin Fab targeting ligands per liposome as described in Examples 1 and 2 (“targeted liposome formulations”) specifically bind to A375.S2 cells and internalize into the same cells in vitro. FIGS. 2A and 2B shown cells that were not treated with drug and, as expected, no evidence of binding or internalization was observed. When the cells were treated with free doxorubicin (i.e., nonliposomal drug) in FIGS. 2C and 2D, drug internalization is evident, however, the diffuse fluorescence pattern suggests the mechanism of drug internalization was nonspecific diffusion of drug across the cell membrane. FIGS. 2E and 2F show cells treated with untargeted liposomes containing doxorubicin. No evidence of binding or internalization of these liposomes was observed. Finally, specific binding and internalization of was observed for the targeted liposome formulation (see FIGS. 2G and 2H) and the fluorescence pattern is marked by regions of high fluorescense intensities on the surface and inside the cytoplasm indicative of liposome internalization under the treatment regime described above.

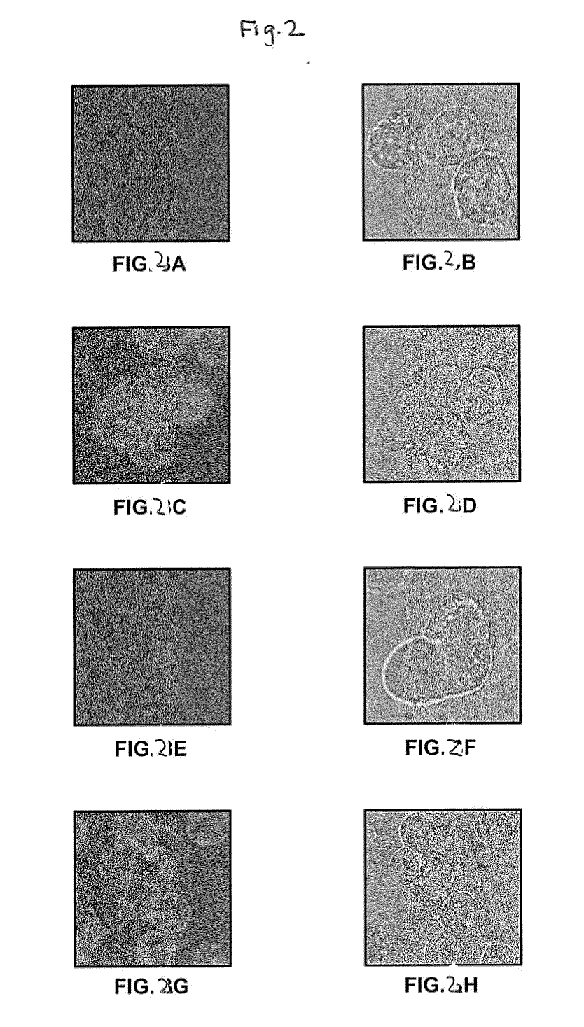

FIG. 3A through 3J show a timecourse study following internalization of the Dextran Alexa Fluor 488 fluorescent marker and doxorubicin (24 hour timepoint only). As the time post-treatment increases, evidence of internalization and penetration into the cytoplasm becomes more clear. More importantly, this data suggests the presence of the fluorescent marker in the cytoplasm may be due to liposome internalization and not fluorescent marker leakage from liposomes followed by diffusion since the fluorescent marker used in this study cannot diffuse across the cell membrane.

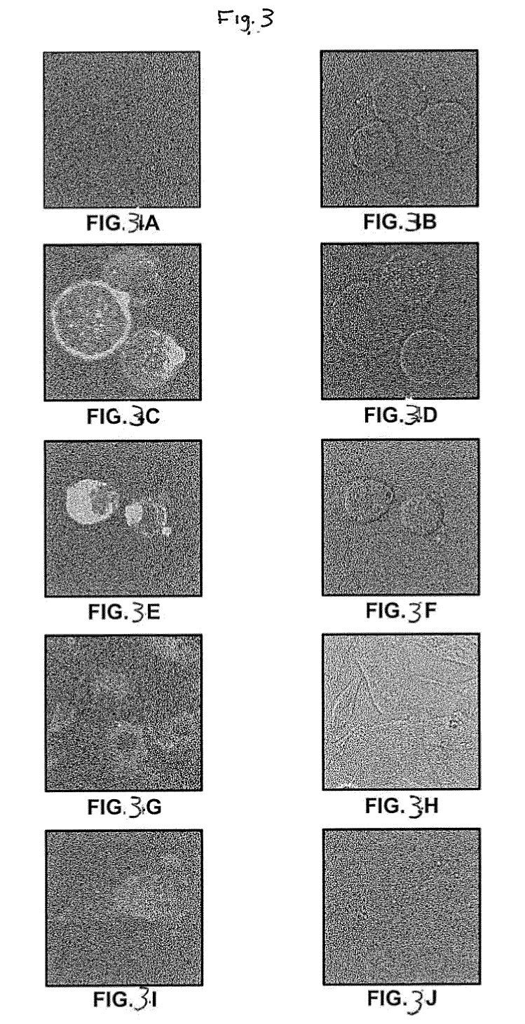

FIGS. 4A through 4H show results from a similar experiment shown in FIG. 2A through 2H. The one change in the experimental conditions was the use of a murine cell line B16.F10 that does not express alpha-V integrins on its cell surface. The purpose of this experiment was to show that liposomes bearing 90:1 alpha-integrin Fab targeting ligands per liposome as described in Examples 1 and 2 only bind to cells expressing alpha-V. The confocal microscopy images demonstrate that this is the case. No binding was observed in this cell line which suggests alpha-V targeted liposomes have a high degree of specificity for alpha-V over-expressing tumor types.

4. Method of Cytotoxicity assay

Cells were harvested by scraping and then resuspended at 37° C. for 1 hour to obtain individualized cells. About 1 million cells of each tumor type were counted and placed in individual centrifugation tubes. The tubes were centrifuged to obtain a cell pellet. The cells were then incubated with the targeted liposome compositions containing doxorubicin for 10 minutes at 37° C., with mild shaking, 140 rpm. Cells were treated with a quantity of liposomes sufficient to give a doxorubicin concentration of 40 μg/mL. After the minute period, 1 mL of washing media (serum free) was added, the cells were vortexed briefly and then centrifuged to obtain a cell pellet. The pellet was resuspended in serum free washing media, vigorously shaking for 10 minutes at 37° C., 440 rpm. After centrifuging again, 1 mL of media containing 10% fetal bovine serum was added. Cells from each tube were seeded on a plate at a concentration of 2000 cell/well, in triplicate for each point. The plate was incubated for 3 and 6 days and then a cell viability assay for cell growth inhibition was conducted.

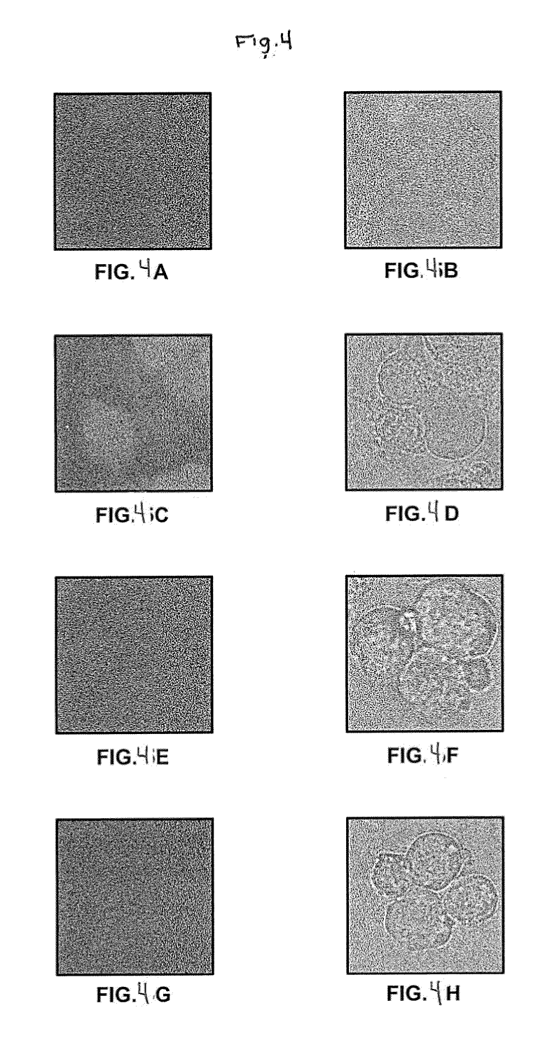

The images (FIGS. 5-8) from the binding study show that alpha-V targeted liposomes specifically bind, and are internalized by the αVβ3/5 integrin positive A375.S2 human melanoma cells. Internalization was time dependent and at longer exposure times, cells internalized a greater number of liposomes. Internalization occurs rapidly, with cells exposed to targeted liposomes for 10 minutes achieving internalization of liposomes into the cell cytoplasm. The presence of liposomes in the nucleus of the cells was also observed in the confocal microscopy images.

The alpha-integrin targeted liposomes displayed specific cytotoxicity toward human αVβ3/5 integrin positive cell lines, including A375.S2, MDA-MB-231, and A2780. As expected, the alpha-V targeted liposomes had no binding to the murine cell line used as a negative control, B16.F10 cell.

The data obtained from the cytotoxicity studies was used to determine molar concentration of each doxorubicin-containing liposomes formulation that produced 50% of the maximum possible response (IC₅₀). IC₅₀ values were determined for doxorubicin in free form, doxorubicin entrapped in liposomes lacking the targeting antibody fragment, and doxorubicin entrapped in liposomes bearing targeting ligands at densities of 40:1 and 90:1 when applied to melanoma tumor cells (A375.S3), breast cancer cells (MDA-MD-231), human ovarian cancer cells (A2780), colon cancer cells (HT29), lung cancer cells (A549), and the non-integrin bearing B16-F10 cells. The values are summarized in Table 2 where the “Increase” corresponds to ratio of IC₅₀ value for liposome-entrapped doxorubicin to the IC₅₀ value for integrin-targeted liposome-entrapped doxorubicin bearing 90:1 ligand:liposome.

TABLE 2
IC₅₀ Values of doxorubicin in various formulations

| | | | | integrin | |
|---|---|---|---|---|---|
| | | | integrin | targeted | |
| | | | targeted | liposome- | |
| | | liposome- | liposome- | entrapped | |
| Tumor Cell | free | entrapped | entrapped dox | dox | |
| Line | dox | dox | 40:1 | 90:1 | Increase |
| melanoma | >200 | 32 | 15 | 7 | 29 |
| AS375.S2 | | | | | |
| breast cancer | >200 | 110 | 45 | 52 | 5 |
| MDA-MS-231 | | | | | |
| ovarian | >200 | 9 | 12 | 7 | 29 |
| cancer A2780 | | | | | |
| colon cancer | >200 | >200 | 180 | >200 | 1.1 |
| HT29 | | | | | |
| Lung cancer | >200 | >200 | >200 | >200 | 1 |
| A549 | | | | | |
| Murine | >200 | 33 | na | >200 | 1 |
| melanoma | | | | | |
| B16-F10 | | | | | |

These data show that certain human tumor derived cell lines are sensitized to the alphaV-targeted liposomes.

### Example 4

### Generation of a Host Cell Line Producing Anti-Alpha-V Fab

The CNTO95 heavy chain signal peptide and variable region from SEQ ID NO: 1 were cloned into expression vector p2032. This vector contained a mouse immunoglobulin promoter, a human IgG1 CH₁ constant region, the first cysteine in the human IgG1 hinge sequence followed by PGK, and a GPT gene for selection of stable integration into the host cell genome. The completed CNTO95 heavy chain Fab expression plasmid, p2324, encoding SEQ ID NO: 3, was co-transfected with the CNTO95 light chain expression plasmid, p2330, into sp2/0 mouse myeloma cells. Cell clones with stable genomic integration of the plasmids were selected based on their resistance to mycophenolic acid in the presence of hypoxanthine. These clones were assayed for Fab expression by ELISA and western blot. The highest expressing clones were subjected to one round of subcloning, with the best subclone expressing 10 ug/ml. This clone, C1021A, was scaled up for further analysis.

The resulting Fab product was designated CNTO 119 and comprised SEQ ID NO: 3 and SEQ ID NO: 2). The C-terminus of the heavy chain bears a single cysteine which can be used effectively for conjugation reactions following mild reduction. The three C-terminal amino acids (PGK) are the same as the C-terminal residues of the full-length IgG1 heavy chain (including CNTO95 heavy chain).

### Example 5

### Preparation of Conjugated sFab-Targeted Stealth Liposomes

A significant portion of the sFab starting material was in the oxidized disulfide form and was subjected to reduction using 10 mM DTE, 40° C., pH 6.0 for 60 minutes to form a free sulfhydryl for conjugation. Excess reductant was removed by passing the reduced sFab material over a desalting column using saline as the running buffer. The pH of the collected sFab fraction was adjusted to pH 6.0 and the protein concentration measured. Since the reduction process produces significant amounts of unwanted by-products (i.e., unassociated light and heavy chains) the sFab material was then subjected to oxidation by introduction of oxygen into the solution to reform the critical disulfide bond between the light and heavy chains that form sFab. Reformation of sFab was monitored by SEC-HPLC. After approximately 4.5 hours of the oxidation reaction, the sFab material was conjugated with MaIPEG-DSPE (5:1 MaIPEG-DSPE:sFab ratio) at room temperature for 1 hour. The solution was quenched for 10 minutes using 1 mM cysteine and run over a desalting column to remove unreacted cysteine. The resulting conjugated sFab material was loaded onto a SEC column (Sephacryl 300) to remove unconjugated protein with PBS as the running buffer. The resulting purity and yield of sFab-conjugate was 95% and 67%, respectively.

sFab-conjugate material was placed over a desalting column to exchange the external buffer to saline. Liposomes containing encapsulated doxorubicin, as described in Example 1 in saline, were inserted with sFab-conjugate at either 60° C. for 1 hour or 37° C. for 48 hour. The amount of sFab added to the liposomes was sufficient to achieve the desired ratio of 15 sFab ligands per liposome. After insertion, the sFab liposome solution was diluted to a final target concentration of 2 mg/mL with saline. The final formulations were in saline. sFab liposome samples were only tested on confocal microscopy to assay bioactivity.

Bioactivity results were negative for formulations inserted at 60° C. while marginal bioactivity of the formulations inserted at 37° C. was observed.

### Example 6

### Comparison of Fab′ Immunoliposomes by Conventional and Oxidation Approaches

In the conventional method, F(ab′)² was reduced under optimal reduction conditions (pH 5.0, 40° C., 9 mM DTE for 30 min). DTE was removed by a desalting column and the protein was added to MaIPEG-DSPE inserted liposomes for surface conjugation. The unconjugated protein was removed from the liposomal fraction using size exclusion chromatography.

For the oxidation method, the Fab′ immunoliposome was prepared as described above in Examples 1-2. F(ab′)² was reduced nearly fully to heavy and light chains (pH 6.5, 13 mM DTE, 40° C. for 60 minutes). After removal of the DTE (and both a desalting and ion exchange column step) the protein underwent a well controlled reoxidation step. At the end of this step, the protein was added to the MaIPEG-DSPE inserted liposomes for surface conjugation. The unconjugated protein was removed from the liposomal fraction using size exclusion chromatography. The results of the comparison are set forth in Table 3.

It can be seen from the data set forth in Table 3, that use of the reoxidation method generates antibody-conjugate purity and yields far greater than the use of conventional methods. It can be expected that antibody-conjugate purity can be achieved by the reoxidation methods disclosed herein in the range of greater than about 80%, in certain embodiments in the range from about 85 to about 97%; and in some embodiments from about 88 to about 95%. Table 3 clearly shows that with Fab-immunoliposome conjugates, purity can be achieved about 88 to about 90%. It can also be expected that antibody-conjugate yields can be achieved by the reoxidation methods disclosed herein that are improvements over conventional methods in the range of about 20 to about 45%, and in certain embodiments in the range from about 20 to about 42%. Table 3 clearly shows that with Fab-immunoliposome conjugates, improvements in yields can be achieved in the range from about 35 to about 45%, and in certain embodiments about 42%.

TABLE 3

| | |
|---|---|
| Conventional Method | REOxidation Method |

| Fab Ratio | FabCG Purity | Yield | FabCG Purity | Yield |
|---|---|---|---|---|
| 15 to 1 | 60 | 38 | 88 | 54 |
| 40 to 1 | 53 | 35 | 89 | 51 |
| 90 to 1 | 45 | 31 | 90 | 47 |

### Example 7

### Comparison of sFab′ Immunoliposomes by Conventional and Oxidation Approaches

The conjugate of the sFab was made and then inserted into the liposomes as in Example 5 using the reoxidation method of the present invention. Conjugates of the sFab were also made and then inserted into liposomes as in conventional approaches without the oxidation method. The results of a comparison of the two approaches are set forth in Table 13.

It can be seen from the data set forth in Table 4, that use of the reoxidation method generates antibody-conjugate purity and yields far greater than the use of conventional methods. It can be expected that antibody-conjugate purity can be achieved by the reoxidation methods disclosed herein in the range of greater than about 80%, in certain embodiments in the range from about 85 to about 97%; and in some embodiments from about 88 to about 95%. Table 4 clearly shows that with sFab-immunoliposome conjugates, purity can be achieved about 95%. It can also be expected that antibody-conjugate yields can be achieved by the reoxidation methods disclosed herein that are improvements over conventional methods in the range of about 20 to about 45%, and in certain embodiments in the range from about 20 to about 42%. Table 4 clearly shows that with Fab-immunoliposome conjugates, improvements in yields can be achieved in the range from about 20 to about 25%, and in certain embodiments about 22%.

TABLE 4

| | |
|---|---|
| Conventional Method | Oxidation Method |

| sFabCG Purity | Yield | sFabCG Purity | Yield |
|---|---|---|---|
| 60 | 55 | 95 | 67 |

### Example 8

### Scale Up and Reproducibility of Fab-Conjugates

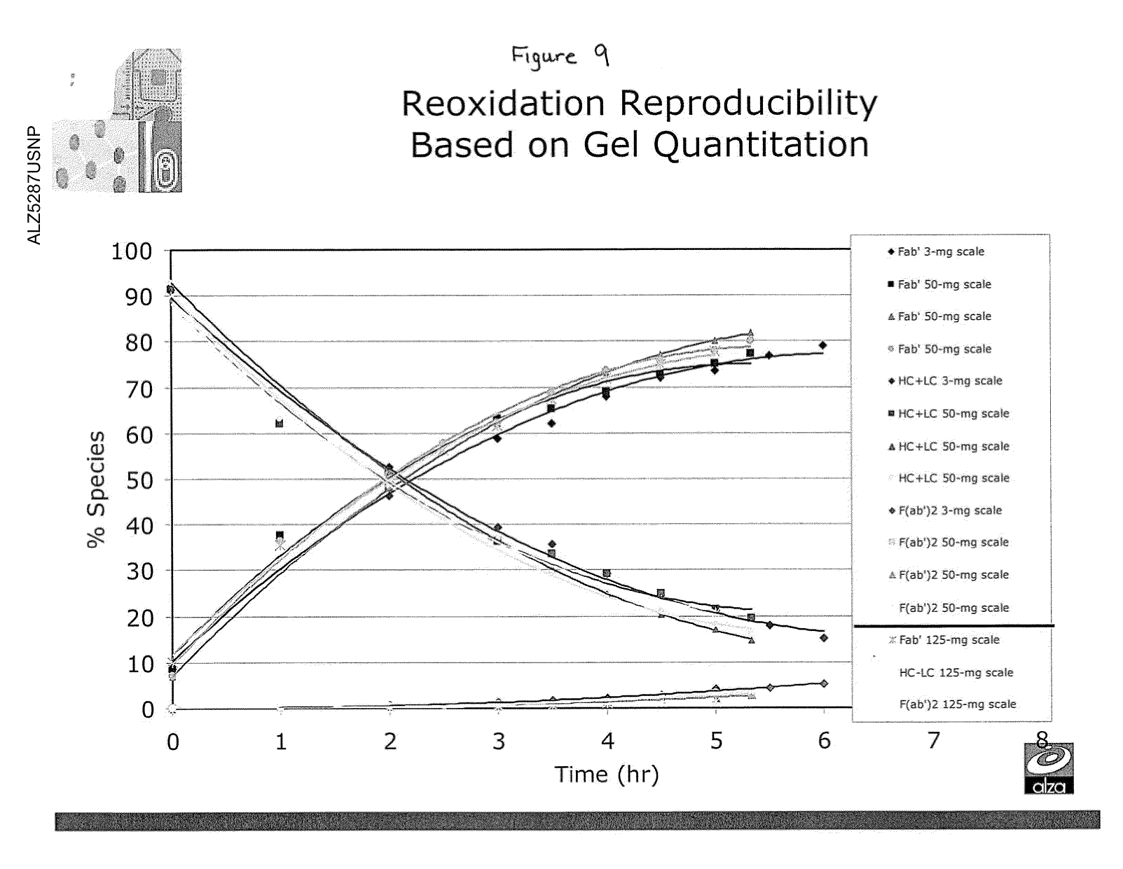

The preparation approaches set forth in Example 2 and developed at the 1.5 mgs was scaled up to 3 to 50 and to 125 mgs of F(ab′)² starting material. As shown in FIGS. 9-12, the reoxidation processes disclosed herein can be successfully scaled up and are reproducible. This is a significant advantage because commercial manufacture of a product is a goal of every manufacturing process.

FIG. 9 shows the reoxidation reproducibility of the process at the 3, 50 and 125 mg scale. During the reoxidation process, a 50 ml sample was taken and blocked with an excess of NEM (n-ethyl maleimide. This reagent reacts very quickly with any free sulfhydryls essentially stopping the reoxidation process for the sample. The progress of the reoxidation process was measured in this way. The samples were later run on SDS-NuPage gels (4-12% bis-tris), stained with Sypro Ruby stain. The species bands on the gel were read on a Typhoon 9400 and quantified by densitometry measurements. The graph shows the amount of each species over the time course for the reoxidation process: the amount of Fab′, HC+LC and F(ab′)2 is shown.

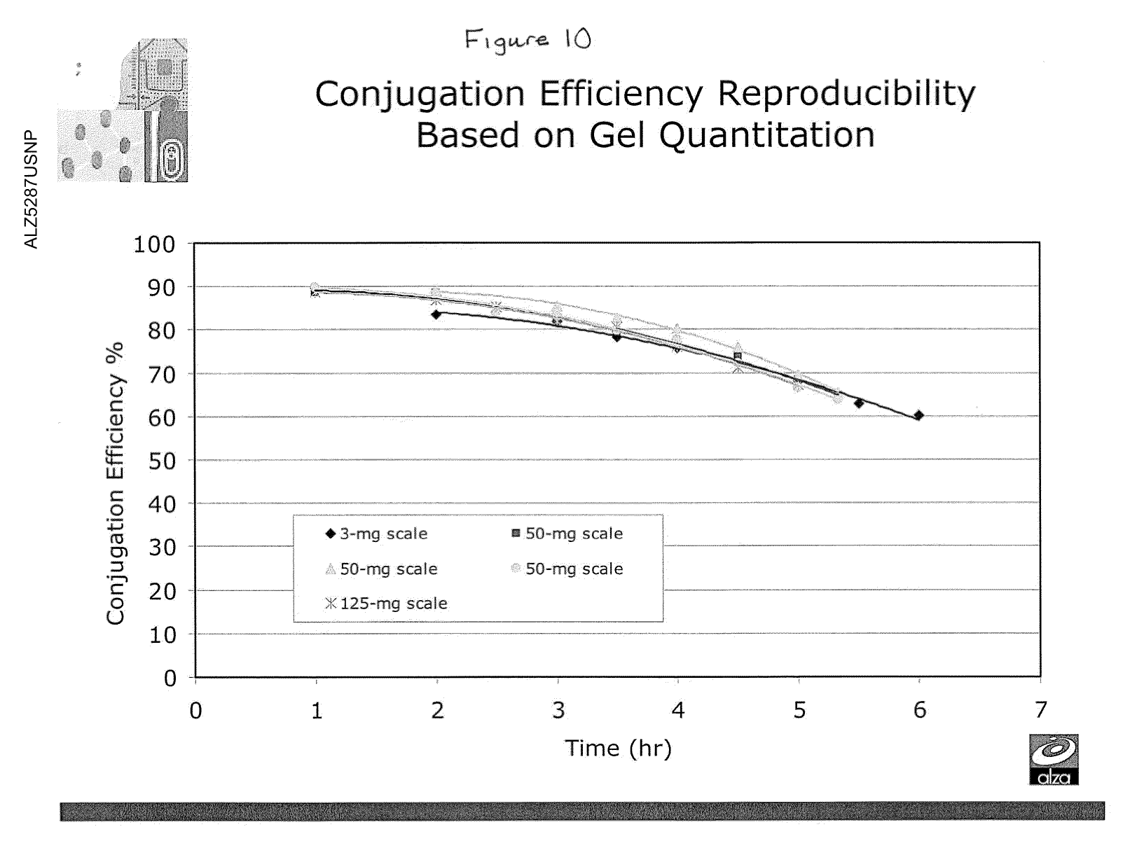

FIG. 10 shows the conjugation efficiency reproducibility of the process at the 3, 50 and 125 mg scale. At the time of conjugation (5 hrs 20 minutes) of the bulk solution, the conjugation efficiency was nearly identical for all runs and scales. Samples were taken throughout the reoxidation process. The samples were later quenched with cysteine to 1 mM and then blocked with iodoacetamide and run on a SDS-NUPage gel and the bands were quantified.

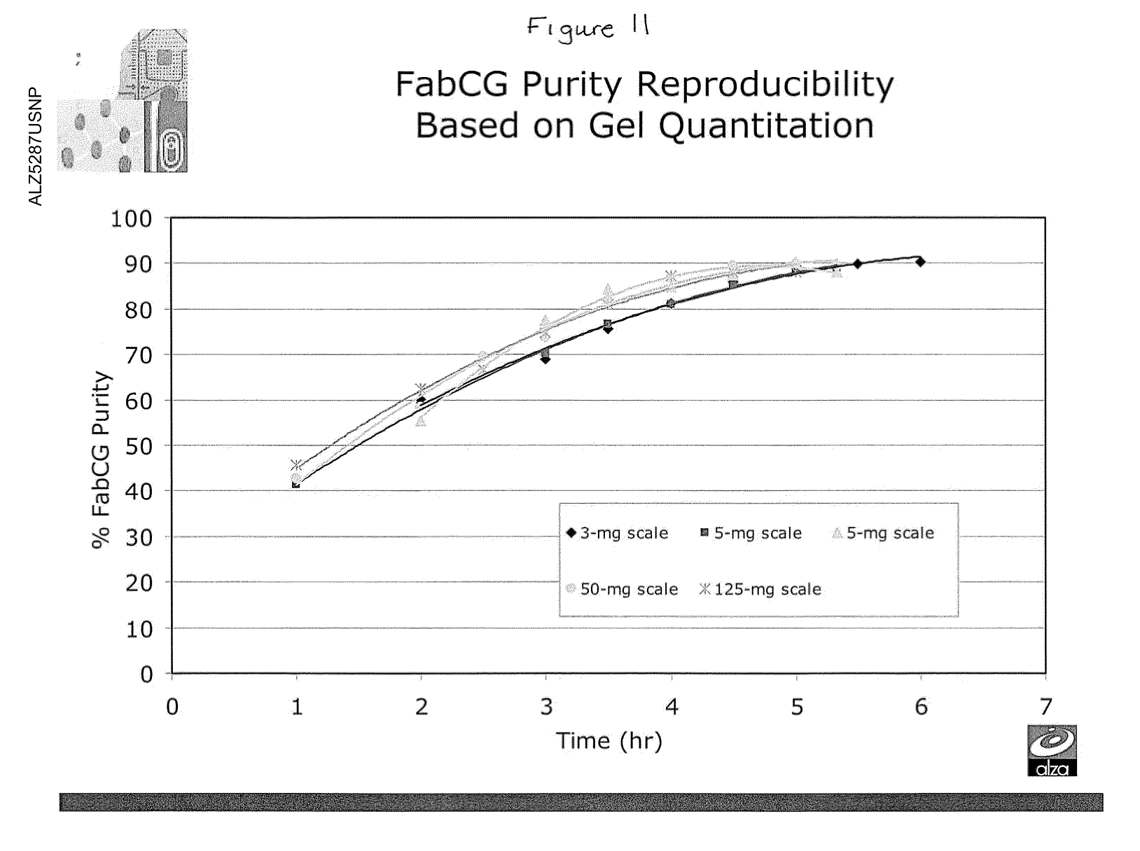

FIG. 11 shows the conjugation purity reproducibility of the process at the 3, 50 and 125 mg scale. At the time of conjugation (5 hrs 20 minutes) of the bulk solution, the conjugation purity was nearly identical for all runs and scales. Samples were taken throughout the reoxidation process. The samples were later quenched with cysteine to 1 mM and then blocked with iodoacetamide and run on a SDS-NUPage gel and the bands were quantified. Purity is defined as the % FabCG divided by the % of all conjugated species present in the sample times 100. Purity increases steadily and then levels off in the low 90s %.

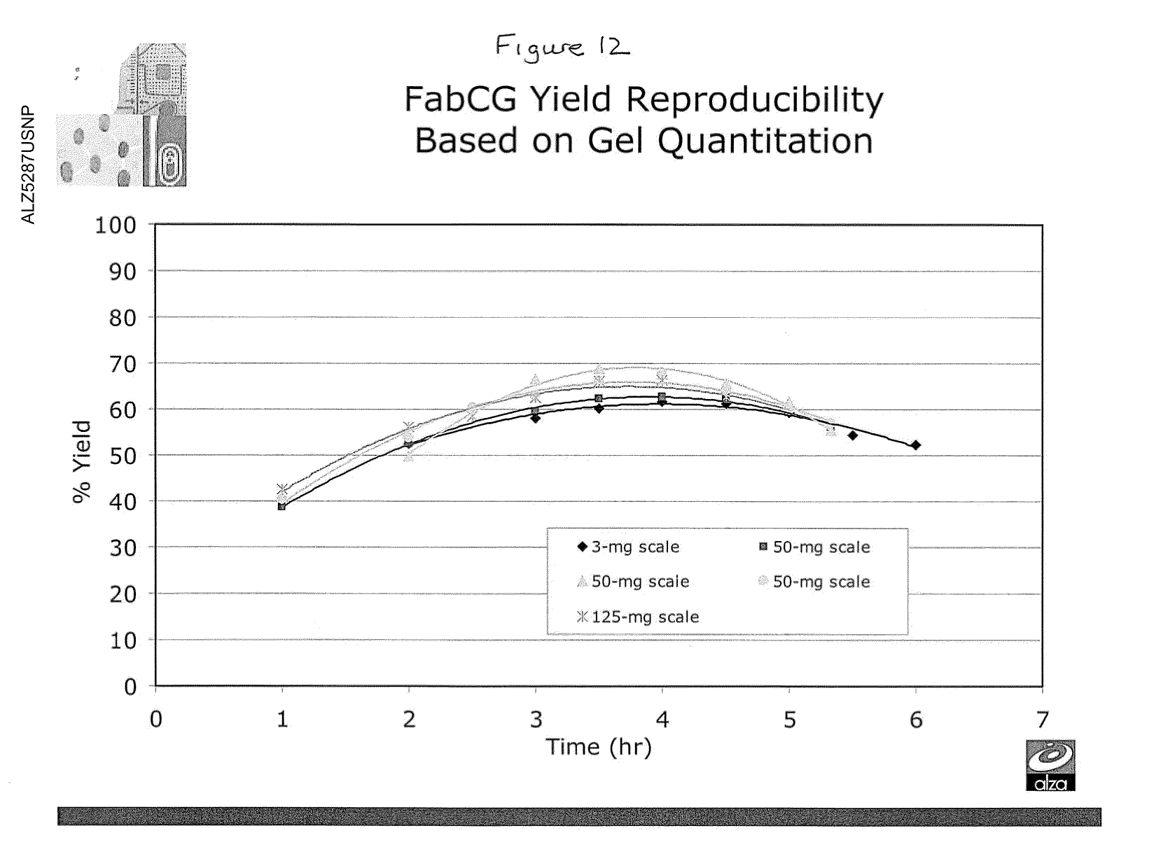

FIG. 12 shows the conjugation yield reproducibility of the process at the 3, 50 and 125 mg scale. At the time of conjugation (5 hrs 20 minutes) of the bulk solution, the conjugation yield was nearly identical for all runs and scales. Samples were taken throughout the reoxidation process. The samples were later quenched with cysteine to 1 mM and then blocked with iodoacetamide and run on a SDS-NUPage gel and the bands were quantified. Yield is defined as the % FabCG divided by all species present in the sample.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A process for preparing an antibody for conjugation to an agent, the process comprising the steps of:
subjecting an antibody comprised of heavy and light chains to reduction conditions prior to conjugation to the agent to break the disulfide bridges between the heavy and light chains;
reforming the disulfide bridges between the heavy and light chains by reoxidation to minimize the presence of heavy and light chains that can be conjugated to the agent; and
conjugating the antibody that results from the reformed heavy and light chains to the agent.

2. The process of claim 1, wherein F(ab)² is subjected to the reduction conditions.

3. The process of claim 1, wherein the antibody that results when the disulfide bridges of the heavy and light chains is reformed is a F(ab).

4. The process of claim 1, wherein the reduction conditions breaks the solvent accessible disulfide bridges of the antibody.

5. The process of claim 1, wherein the process generates the antibody-conjugate in good yields and purity.

6. The process of claim 5, wherein the purity of the antibody-conjugate is greater than about 80%.

7. The process of claim 5, wherein the yield of the antibody-conjugate is in the range from about 20 to about 45%.

8. The process of claim 5, wherein the antibody that is subjected to reduction conditions is an antibody that has specific binding activity for alpha-V-integrin receptors.

9. The process of claim 8, wherein the antibody is a F(ab)² that is an immunoglobulin that has specific binding activity for alpha-V-beta-3 (αvβ3) integrin receptors.

10. The process of claim 8, wherein the antibody is a F(ab)² that is an immunoglobulin that has specific binding activity for alpha-V-beta-5 (αvβ5) integrin receptors.

11. The process of claim 8, wherein the antibody is a F(ab)² that is an immunoglobulin that has specific binding activity for alpha-V-beta-3 (αvβ3) and alpha-V-beta-5 (αvβ5) integrin receptors.

12. The process of claim 1, wherein the resulting antibody is conjugated to a therapeutic agent.

13. The process of claim 1, wherein the resulting antibody is conjugated to a lipidic microparticle.

14. The process of claim 13, wherein the resulting antibody is conjugated to a liposome.

15. The process of claim 13, wherein the resulting antibody is conjugated to a liposomal component.

16. The process of claim 1, wherein the reduction conditions comprised the use of DTE.

17. The process of claim 16, further comprising the step of removing the DTE.

18. The process of claim 1, wherein the oxidation step is performed in the presence of trace amounts of Cu⁺².

19. The process of claim 1, further comprising the step of adding a chelating agent to the preparation after the heavy and light chains reform to minimize further oxidation.

20. The process of claim 19, wherein the chelating agent is EDTA.
